(19) United States
(12) Patent Application Publication
(10) Pub. No.: US 2025/0231173 A1
(43) Pub. Date: Jul. 17, 2025

(54) ARTICLES AND METHODS FOR PLASMA SEPARATION

(71) Applicant: Trustees of Tufts College, Medford, MA (US)

(72) Inventors: Charles R. Mace, Winchester, MA (US); Giorgio Gianini Morbioli, Somerville, MA (US); Keith Baillargeon, Medford, MA (US)

(73) Assignee: Trustees of Tufts College, Medford, MA (US)

(21) Appl. No.: 18/705,119

(22) PCT Filed: Oct. 28, 2022
(86) PCT No.: PCT/US2022/048205

§ 371 Date: Apr. 26, 2024

Related U.S. Application Data
(60) Provisional application No. 63/273,740, filed on Oct. 29, 2021.
(60) Provisional application No. 63/292,274, filed on Dec. 21, 2021.

(51) Int. Cl. G01N 33/49; G01N 33/50; G01N 33/80

(52) U.S. Cl. CPC G01N 33/491; G01N 33/5002; G01N 33/80

(57) ABSTRACT

Articles and methods to separate blood cells from plasma are generally provided. In some embodiments, an article comprises an absorbent layer comprising a sample collection region laterally spaced from and fluidically connected with filters. The sample collection region may be removable, in some embodiments. In some embodiments, an article comprises a first filter and a second, filter configured to separate blood cells from plasma positioned between the environment external to the article and the first filter. In the context of the present disclosure, it has been recognized that the articles and methods described herein can be used to passively separate plasma with a high purity from whole blood.

### RELATED APPLICATIONS

This application claims priority under 35 U.S.C. § 119(e) to U.S. Provisional Application No. 63/273,740, filed Oct. 29, 2021, and entitled “ARTICLES AND METHODS FOR PLASMA SEPARATION,” and to U.S. Provisional Application No. 63/292,274, filed Dec. 21, 2021, and entitled “ARTICLES AND METHODS FOR PLASMA SEPARATION,” each of which is incorporated herein by reference in its entirety for all purposes.

### GOVERNMENT SPONSORSHIP

This invention was made with government support under grant EB027049 awarded by the National Institutes of Health. The government has certain rights in the invention.

### TECHNICAL FIELD

Articles and methods related to blood separation are generally provided.

### SUMMARY

Articles and methods to separate blood cells from plasma are generally provided. The subject matter of the present invention involves, in some cases, interrelated products, alternative solutions to a particular problem, and/or a plurality of different uses of one or more systems and/or articles.

In one aspect, an article configured to separate blood cells from plasma is provided. In some embodiments, the article configured to separate blood cells from plasma comprises: a first filter configured to retain blood cells; a second filter configured to retain blood cells, wherein the second filter is disposed beneath the first filter, and wherein the first and second filters are positioned such that a sample comprising separated blood cells can be recovered therefrom; and an absorbent layer comprising a porous, absorbent material, wherein: the absorbent layer is disposed beneath the second filter, the absorbent layer comprises a sample collection region fluidically connected with and laterally spaced from the second filter, and the sample collection region is configured to receive plasma from which blood cells have been separated from the second filter.

In another aspect, an article configured to separate blood cells from plasma is provided. In some embodiments, the article configured to separate blood cells from plasma, comprises: a filter configured to retain blood cells; and an absorbent layer comprising a porous, absorbent material, wherein: the absorbent layer is disposed beneath the filter, the absorbent layer comprises a sample collection region laterally spaced from the filter, the absorbent layer comprises a channel fluidically connecting the filter to the sample collection region, the sample collection region is configured to receive plasma from which blood cells have been separated, and the sample collection region is laterally bounded in a plane of the channel by a boundary and a terminus of the channel, the boundary comprises a section having a distance from the terminus of the channel, and a standard deviation of a distance from the terminus of the channel to the section is less than or equal to 30% of an average distance from a terminus of the channel to the section, and wherein the section makes up greater than or equal to 15% of the boundary.

In yet another aspect, a method is provided. In some embodiments, the method comprises: passing a blood sample comprising blood cells and plasma to an absorbent layer through a first filter and a second filter to separate at least a portion of the blood cells from the plasma; and transporting the plasma laterally within the absorbent layer to a sample collection region that is laterally spaced from the first and second filters.

In still another aspect, a method is provided. In some embodiments, the method comprises: passing a blood sample comprising blood cells and plasma to an absorbent layer through a filter to separate at least a portion of the blood cells from the plasma; and transporting the plasma laterally within the absorbent layer to a sample collection region, wherein the sample collection region is laterally bounded in a plane of the layer by a boundary and a terminus of a channel, wherein the boundary comprises a section having a relatively constant distance from a terminus of a channel, wherein a standard deviation of a distance from the terminus of the channel to the section is less than or equal to 50% of an average distance from the terminus of the channel to the section, and wherein the section makes up greater than or equal to 15% of the boundary.

In one aspect, an article configured to separate blood cells from plasma is provided. According to some embodiments, the article comprises: a filter configured to retain blood cells; and an absorbent layer comprising a porous, absorbent material, wherein: the absorbent layer is disposed beneath the filter, the absorbent layer comprises a sample collection region laterally spaced from the filter, the absorbent layer comprises a channel fluidically connecting the filter to the sample collection region, the sample collection region is configured to receive plasma from which blood cells have been separated, the sample collection region is laterally bounded in a plane of the channel by a boundary and a terminus of the channel, the sample collection region comprises a back portion, and the back portion is closer to a portion of the channel directly upstream from the channel terminus than it is to the channel terminus.

In another aspect, a method is provided. In some embodiments, the method comprises: passing a blood sample comprising blood cells and plasma to an absorbent layer through a filter to separate at least a portion of the blood cells from the plasma; transporting the plasma through a channel within the absorbent layer to a sample collection region; and transporting at least a portion of the plasma into a back portion of the sample collection region, wherein the back portion is closer to a portion of the channel directly upstream from the channel terminus than it is to the channel terminus.

In still another aspect, an article for collecting both whole blood and plasma is provided. According to some embodiments, the article comprises: a first layer comprising a sample inlet; a fluid distribution layer disposed beneath the sample inlet; a filter configured to retain blood cells, fluidically connected with and disposed beneath the fluid distribution layer; and a second, absorbent layer disposed beneath the filter and comprising a porous, absorbent material, wherein: the second, absorbent layer comprises a plasma collection region fluidically connected with the filter and configured to receive fluid from the filter, and the second, absorbent layer comprises a whole blood collection region fluidically isolated from the plasma collection region in the second, absorbent layer and configured to receive fluid directly from the fluid distribution layer.

In yet another aspect, a method is provided. According to some embodiments, the method comprises: passing a blood sample comprising blood cells and plasma through a sample inlet positioned in a first layer; passing the blood sample received from the sample inlet through a fluid distribution layer; passing a first portion of the blood sample received from the fluid distribution layer through a filter, thereby separating blood cells from the plasma in the first portion of the blood sample; transporting the plasma from the first portion of the blood sample into a plasma collection region positioned in a second, absorbent layer comprising a porous, absorbent material; and transporting a second portion of the blood sample directly from the fluid distribution layer to a whole blood collection region positioned in the second, absorbent layer.

In another embodiment, an article for collecting both whole blood and plasma is provided. According to some embodiments, the article comprises: a first layer comprising a sample inlet and a whole blood collection region; a fluid distribution layer disposed beneath the sample inlet; a filter configured to retain blood cells, fluidically connected with and disposed beneath the fluid distribution layer; and a second, absorbent layer disposed beneath the filter and comprising a porous, absorbent material, wherein: the second, absorbent layer comprises a plasma collection region fluidically connected with the filter and configured to receive fluid from the filter, and the whole blood collection region is fluidically isolated from the inlet in the first layer and configured to receive fluid directly from the fluid distribution layer.

In one embodiment, a method is provided. According to some embodiments, the method comprises: passing a blood sample comprising blood cells and plasma through a sample inlet positioned in a first layer; passing the blood sample received from the sample inlet through a fluid distribution layer; passing a first portion of the blood sample received from the fluid distribution layer through a filter, thereby separating blood cells from the plasma in the first portion of the blood sample; transporting the plasma from the first portion of the blood sample into a plasma collection region positioned in a second, absorbent layer comprising a porous, absorbent material; and transporting a second portion of the blood sample directly from the fluid distribution layer to a whole blood collection region positioned in the first layer.

In another embodiment, an article is provided. According to some embodiments, the article comprises: a filter configured to retain blood cells; a first, absorbent layer comprising a first porous, absorbent material; and a second, absorbent layer comprising a second porous, absorbent material, wherein: the first, absorbent layer is disposed beneath the filter, the second, absorbent layer is disposed above or below the first, absorbent layer, the first, absorbent layer comprises a sample collection region fluidically connected with and laterally spaced from the filter, the first, absorbent layer comprises a channel fluidically connecting the filter to the sample collection region, the sample collection region is laterally bounded in a plane of the channel by a boundary and a terminus of the channel, the second, absorbent layer comprises an overflow region in fluidic communication with the sample collection region, the overflow region comprises a receiving portion that overlaps the sample collection region at an overlap portion of the sample collection region, the overlap portion extends inwards from the boundary of the sample collection region, the overflow region extends outwards from the receiving portion thereof, and the sample collection region further comprises a non-overlap portion that does not overlap the overflow region.

In yet another embodiment, a method is provided. According to some embodiments, the method comprises: passing a blood sample comprising blood cells and plasma to a first, absorbent layer through a filter to separate at least a portion of the blood cells from the plasma; transporting the plasma laterally within the first, absorbent layer to a sample collection region, wherein the sample collection region is laterally bounded in a plane of the layer by a boundary and a terminus of a channel, and transporting excess plasma out of the sample collection region to a receiving portion of an overflow region positioned in a second, absorbent layer disposed above or below the first, absorbent layer, wherein the overflow region extends outwards from the receiving portion thereof, and wherein the sample collection region comprises a non-overlap portion that does not overlap the overflow region.

In still another embodiment, an article is provided. According to some embodiments, the article comprises: a filter configured to retain blood cells; and an absorbent layer comprising a first porous, absorbent material; wherein: the absorbent layer is disposed beneath the filter, the absorbent layer comprises a sample collection region fluidically connected with and laterally spaced from the filter, the absorbent layer comprises a channel fluidically connecting the filter to the sample collection region, the sample collection region is laterally bounded in a plane of the channel by a boundary and a terminus of the channel, the absorbent layer comprises an overflow region in fluidic communication with the sample collection region via interstices in the boundary of the sample collection region.

In one embodiment, a method is provided. According to some embodiments, the method comprises: passing a blood sample comprising blood cells and plasma to an absorbent layer through a filter to separate at least a portion of the blood cells from the plasma; transporting the plasma laterally within the absorbent layer to a sample collection region, wherein the sample collection region is laterally bounded in a plane of the layer by a boundary and a terminus of a channel, and transporting excess plasma out of the sample collection to an overflow region in the absorbent layer and separated from the sample collection region by interstices in the boundary, through which the excess plasma is transported.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1B present cross-sectional schematic illustrations of exemplary articles comprising a filter and an absorbent layer, according to some embodiments;

FIG. 2 presents a cross-sectional schematic illustration of an exemplary article comprising filters and an absorbent layer, according to some embodiments;

FIGS. 3-10 present top view schematic illustrations of exemplary absorbent layers, according to some embodiments;

FIGS. 11A-11D present cross-sectional schematic illustrations of exemplary articles comprising a fluid distribution layer and a whole blood collection region, according to some embodiments;

FIGS. 12A-12C present exploded perspective schematic illustrations of exemplary articles comprising a fluid distribution layer and a whole blood collection region, according to some embodiments;

FIG. 13A presents a top-view schematic illustration of an exemplary overflow region, according to some embodiments;

FIG. 13B presents a top-view schematic illustration of an exemplary overflow region overlaid with an absorbent layer comprising a sample collection region, according to some embodiments;

FIG. 13C presents a top view schematic illustration of an exemplary absorbent layer, according to some embodiments;

FIG. 13D presents a top view schematic illustration of an exemplary absorbent layer, according to some embodiments;

FIG. 14 presents an exploded perspective schematic illustration of an exemplary article comprising first, absorbent layer and a second, absorbent layer comprising an overflow region, according to some embodiments;

FIG. 15 presents an exploded perspective schematic illustration of an exemplary article comprising absorbent layers and filters, according to some embodiments;

FIG. 16 presents a schematic illustration of separation of a blood sample in an exemplary article, according to some embodiments;

FIG. 17A presents a schematic method of separation of a blood sample, according to some embodiments;

FIG. 17B presents a schematic method of separation of a blood sample, according to some embodiments;

FIG. 18 presents an exploded perspective schematic illustration of an exemplary article comprising absorbent layers and filters, according to some embodiments;

FIGS. 19A-19G and 20A-20B show images of exemplary articles, according to some embodiments;

FIG. 21 is a plot showing the plasma yield as a function of hematocrit percentage for an exemplary article, according to some embodiments;

FIG. 22 presents a photograph of non-limiting articles comprising multiple sample collection regions, according to some embodiments;

FIGS. 23A and 23B present information regarding the mass of plasma collected within plasma collection regions, according to some embodiments;

FIGS. 24A and 24B present photographs of a non-limiting article, according to some embodiments;

FIGS. 25A and 25B present photographs of a non-limiting article, according to some embodiments; and

FIGS. 26A and 26B present plasma volumes collected within sample collection regions of non-limiting articles, according to some embodiments.

### DETAILED DESCRIPTION

Articles and methods related to blood separation are generally provided. Some articles described herein may be configured to receive a fluid sample. The articles may have a design that spatially (e.g., laterally) separates plasma from blood cells. This may be accomplished in two steps: a vertical filtration step, in which blood cells are removed from blood and retained on one or more filters, and a lateral transport step, in which plasma from which blood cells have been removed is transported laterally away from the filter(s). As a non-limiting example, in some embodiments, a blood sample placed on a first filter of the article is passed to an absorbent layer through a filter and laterally transmitted to a sample collection region. The sample collection region may be configured to be removed from the article (e.g., using tweezers) for later analysis of the sample therein. One advantage of the embodiments disclosed herein is that the plasma and the filtered blood cells may be analyzed separately, at least in part due to their spatial and/or lateral separation. This may allow each to be analyzed at a relatively higher concentration than would be observed in whole blood.

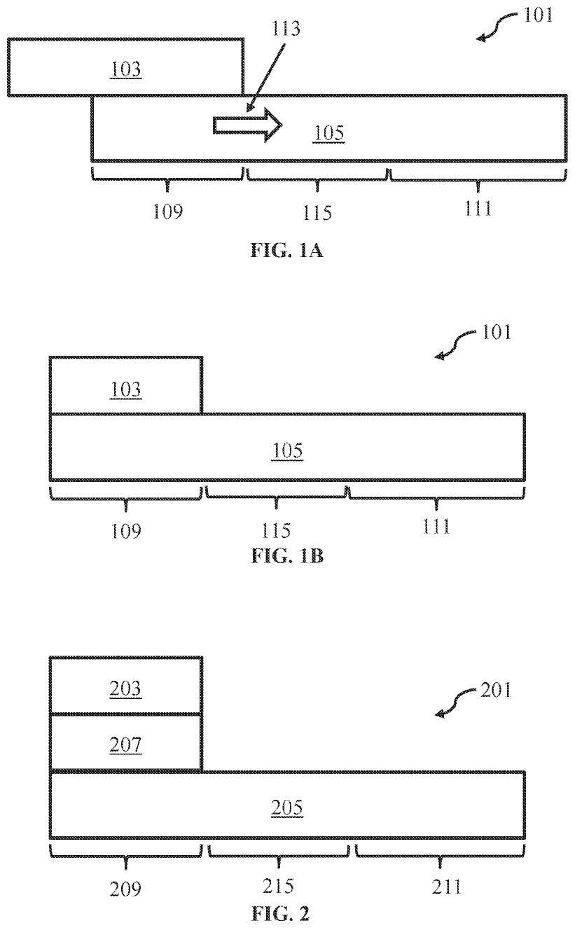

In one aspect, the present disclosure is directed towards an article. The article may be configured to separate components of a fluid (e.g., a blood sample). For example, the article may be configured to separate blood cells (e.g., red blood cells, white blood cells, platelets) from plasma. The article may comprise more than one layer. For example, in some embodiments, the article comprises one or more filters, absorbent layers, and/or adhesive layers. In some embodiments, the article comprises a filter and an absorbent layer. For example, FIG. 1A presents a cross-sectional schematic illustration of article 101, comprising filter 103 and absorbent layer 105, according to some embodiments.

In some embodiments, the article is configured such that the absorbent layer can draw a fluid (e.g., a blood sample) through a filter. For example, the absorbent layer may be porous. The absorbent layer may be disposed beneath the filter. By way of example, with reference to FIG. 1A, absorbent layer 105 may be disposed beneath filter 103 as shown in FIG. 1A, such that at least part of the filter overlaps the absorbent layer. The filter may be disposed on a filter reception region of the absorption layer. For example, in the example of FIG. 1A, the filter is disposed on filter reception region 109 of the absorption layer. In some embodiments, as shown in FIG. 1B, the entire filter overlaps the absorbent layer. The absorbent layer may be fluidically connected to the filter, in some embodiments. For example, the absorbent layer may directly contact the filter, or may be connected to the filter via an intervening layer permitting the passage of fluid. It should be noted that FIGS. 1A-1B present a transverse cross-section of the article, and that the channel, sample collection region, and filter reception region may have different lateral profiles that are not represented in these figures. Further Figures showing these lateral profiles are presented and described later in the application.

According to some embodiments, the article comprises a first filter and a second filter. For example, FIG. 2 presents article 201 comprising first filter 203 and second filter 207. In some embodiments, the absorbent layer is disposed beneath the second filter. For example, in FIG. 2, absorbent layer 205 is disposed beneath second filter 207. Like FIGS. 1A-1B, FIG. 2 presents a transverse cross-section of the article, and the channel, sample collection region, and filter reception region have different lateral profiles that are not represented in these figures. For articles having the design shown in FIG. 2, further Figures showing these lateral profiles are also presented and described later in the application.

The layers of the article may be free-standing, as shown in the examples of FIGS. 1A-2. However, the layers of the article may instead be supported by a supporting structure. The layers and supporting structures of the article are described in greater detail below.

In another aspect, a method is provided. In some embodiments, the method comprises passing fluid (e.g., a blood sample) through an article. Non-limiting examples of fluid samples that may be analyzed in the articles described herein include fluids of biological origin, such as blood (e.g., whole blood) and fluids derived from blood (e.g., plasma), cerebrospinal fluid, tissue biopsies, milk, wound exudate, saliva, tears, or urine.

In some embodiments, the blood sample is whole blood. In some embodiments, the blood sample is undiluted blood from a subject. In some embodiments, the subject is an animal, such as a mammal. In some embodiments, the subject is a human. In some embodiments, the article comprises an anti-coagulant (e.g., ethylenediaminetetraacetic acid (EDTA) and/or heparin), such as a dried anti-coagulant.

A blood sample may comprise blood cells and plasma. The method may separate blood cells from plasma (e.g., using filters). For instance, in some embodiments, the article is configured to separate blood cells from plasma by passing the blood sample through the filter(s) of the article. In the case of articles configured to receive samples comprising blood, it may be desirable for the samples to be relatively rich in certain portions of blood and relatively poor in (or lacking entirely) others. For instance, it may be desirable for a filter to be configured to separate blood cells from plasma. Some advantageous filters may be configured to allow a relatively high proportion of the plasma in blood to pass through the filter, and may also be configured to retain a relatively high proportion of the cells in blood on the filter. Other types of filters (e.g., that filter blood in a different manner, that are configured to filter one or more components of another type of fluid sample) may also be employed. In some embodiments, it is desirable to use a relatively small volume of blood in the article. For example, it may be desirable to use a volume of less than or equal to 200 microliters, less than or equal to 180 microliters, less than or equal to 150 microliters, less than or equal to 140 microliters, less than or equal to 130 microliters, less than or equal to 120 microliters, less than or equal to 110 microliters, less than or equal to 100 microliters or less of blood in the article. One advantage of the articles and methods described herein may be the reduction in the blood volume required to produce viable plasma and/or blood cell samples for testing.

In some embodiments, a fluid (e.g., a blood sample) is passed to an absorbent layer. The absorbent layer may be configured to transport fluid spatially. For example, the absorbent layer may be configured to transport fluid laterally. In some embodiments, the article may comprise a filter, and the filter may be configured to transport a fluid spatially away from the filter. For example, in some embodiments, an article may comprise a filter and may be configured to transport a fluid sample laterally away from the filter. As an illustrative example, FIG. 1A includes arrow 113, representing transportation of fluid laterally from filter 103. Without wishing to be bound by theory, the spatial transport (e.g., the lateral transport of) the fluid may be driven by capillary action. For example, the lateral transport of the fluid may arise from wicking of the fluid laterally by a comparatively fluid-free portion of an absorbent layer (e.g., a sample collection region).

In some embodiments, the absorbent layer is configured to transport fluid to a sample collection region. For example, in the examples of FIGS. 1A-1B, absorbent layer 105 is configured to transport fluid laterally from filter 103 to sample collection region 111. The fluid may be transported through a channel. For example, in the example of FIGS. 1A-1B, the fluid may be transported through channel 115. In some embodiments, the fluid is transported from the filter reception region of the absorbent layer. For example, in the examples of FIGS. 1A-1B, the fluid may be transported from filter reception region 109 of absorbent layer 105, where it is initially introduced to the absorbent layer via filter 103.

In some embodiments, an article comprises a filter configured to separate blood cells from plasma, and is configured to transport plasma passed through the filter and away from the filter. Some methods may comprise forming samples comprising plasma by passing a blood sample through a filter configured to separate blood cells from plasma, retaining at least a portion of the cells on a first side of the filter (e.g., a side of the filter closer to an environment external to the article), and transporting at least a portion of the plasma away from the filter.

In some embodiments, one or more of the layers of the article is an absorbent layer. The absorbent layer, according to some embodiments, comprises a porous, absorbent material, as described in greater detail below. The porous, absorbent material may, upon exposure to a fluid sample, wick the fluid sample into the layer and/or wick the fluid sample through the layer. When a layer comprises a channel comprising a porous, absorbent material, the porous, absorbent material may wick the fluid sample into the channel and/or through the channel. In some embodiments, a fluid may flow into and/or through a porous, absorbent material (e.g., a porous, absorbent material present in a channel) due to capillarity (capillary action) or by wicking. In some embodiments, a porous, absorbent material will, upon exposure to a fluid sample (e.g., a fluid sample of interest, a fluid sample for which it is absorbent), transport the fluid sample into the interior of the porous, absorbent material (i.e., the fluid sample may penetrate into the interior of the material in which the pores are positioned, such as into the interior of fibers making up a porous, absorbent material that comprises fibers). In some embodiments, a porous, absorbent material will, upon exposure to a fluid sample, experience an increase in mass due to the fluid sample absorbed therein. It should be understood that some layers comprising porous absorbent materials may have one or more of the properties described above with respect to porous, absorbent materials.

In some embodiments, the absorbent layer comprises a sample collection region. For example, in FIGS. 1A-1B, absorbent layer 105 comprises sample collection region 111, and in FIG. 2, absorbent layer 205 comprises sample collection region 211.

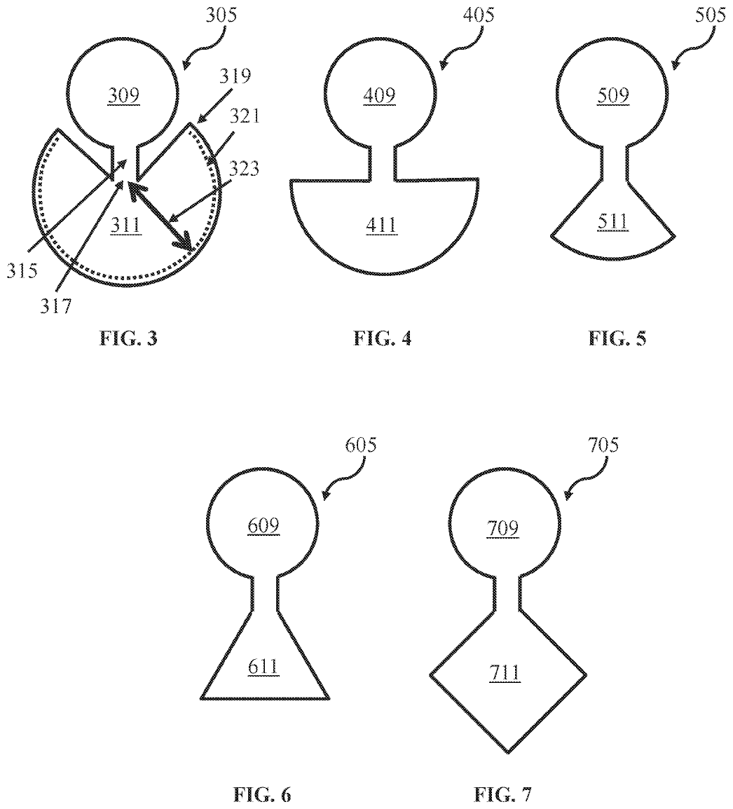

The sample collection region may be laterally spaced from the filter(s). For example, in the embodiments depicted in FIG. 3, sample collection region 311 is laterally spaced from filter reception region 309. The sample collection region may be fluidically connected with the filter(s). For example, the absorbent layer may be fluidically connected with the filter(s) via a channel. As illustrated in FIG. 3, sample collection region 311 may be fluidically connected to filter(s) (not shown) via channel 315 and filter reception region 309. The sample collection region may be configured to receive a fluid (e.g., plasma, or a sample with reduced blood cells). For example, the sample collection region may be configured to receive the fluid via the fluidic connection to the filter(s) (e.g., the first filter and/or the second filter).

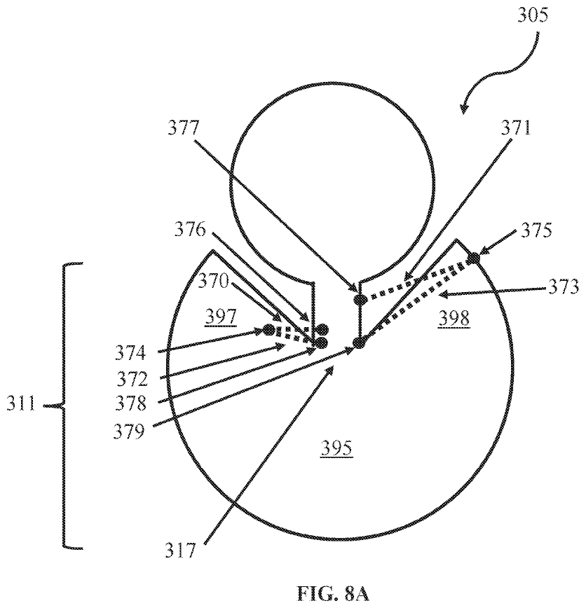

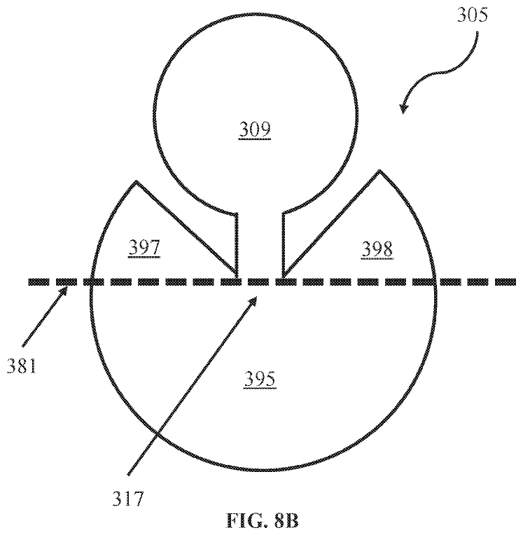

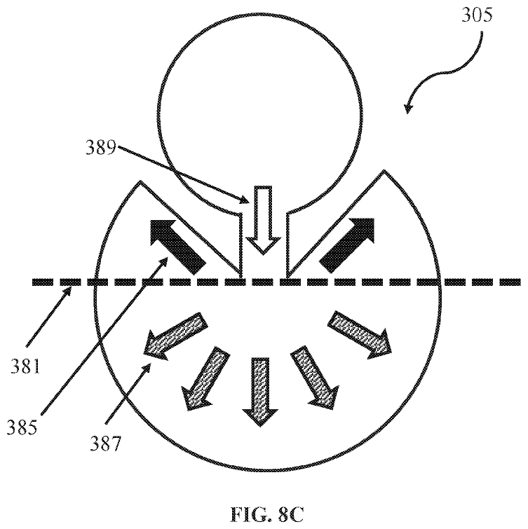

In the context of the present disclosure, it has been inventively recognized that sample collection regions having a relatively uniform distance between a terminus of the channel and a portion of a boundary of the sample collection region may be advantageous. For example, sample collection regions with this property may achieve relatively rapid flow of relatively pure plasma into the sample collection region, as described in greater detail below. FIGS. 3-7 present top-view schematic illustrations of absorbent layers, comprising different, exemplary sample collection regions. The sample collection region may have any appropriate form. To provide a few, non-limiting examples, the sample collection region may have the form of a circular sector (e.g., defined by an angle around a circular center), a polygon (e.g., a triangle, a square), a circle, an ellipse, or any of a variety of other forms. For example, FIG. 3 presents exemplary absorbent layer 305 that comprises a filter reception region 309 and sample collection region 311, in the form of a circular sector defined by an angle greater than 180°, according to some embodiments. FIG. 4 presents exemplary absorbent layer 405 that comprises filter reception region 409 and sample collection region 411, in the form of a semicircular sector, according to some embodiments. FIG. 5 presents exemplary absorbent layer 505 that comprises filter reception region 509 and sample collection region 511 in the form of a circular sector defined by an angle less than 180°, according to some embodiments. FIG. 6 presents exemplary absorbent layer 605 that comprises filter reception region 609 and sample collection region 611 in the form of an isosceles triangle, according to some embodiments. FIG. 7 presents exemplary absorbent layer 705 that comprises filter reception region 709 and sample collection region 711, in the form of a square, according to some embodiments.

Sample collection regions in the form of circular sectors may be advantageous because they provide a relatively uniform distance between a terminus of the channel and a portion of the boundary of the sample collection region. It should be understood that sample collection regions in the form of circular sectors need not be centered on the terminus of the channel. In general, the form of the sample collection region refers to a profile of the sample collection region, rather than a geometrically perfect shape. For example, the form of the sample collection region may be distorted at a terminus of a channel. Thus, sample collection region 311, shown in FIG. 3, has the form of a circular sector but is not a geometrically perfect shape—it is distorted, at least where it meets lateral terminus 317 of channel 315.

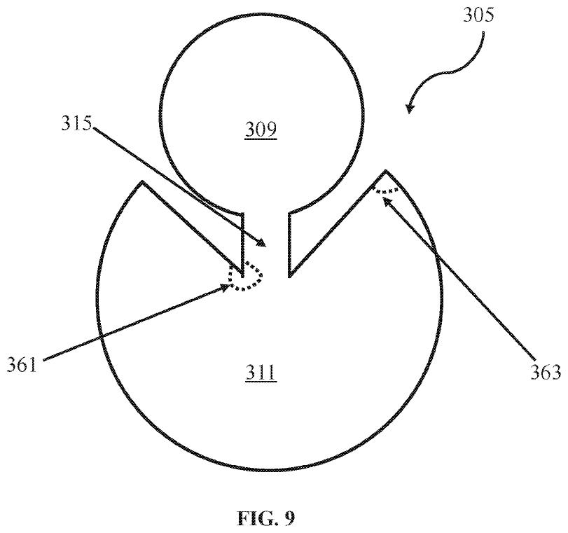

In some embodiments, a sample collection region comprises one or more portions. As one example, a sample collection region may comprise a back portion. The back portion of the sample collection region may be closer to a portion of the channel directly upstream from the channel terminus than it is to the channel terminus. FIG. 8A presents a non-limiting, schematic illustration of absorbent layer 305 comprising sample collection region 311 which itself comprises back portions 397 and 398. As can be seen in FIG. 8A, point 374 of back portion 398 is closer to point 376 upstream of channel terminus 317 than it is to point 378 at channel terminus 317, as can be seen by comparing the length of distance 370 (connecting points 374 and 376) and distance 372 (connecting points 374 and 378).

Without wishing to be bound by any particular theory, it is believed that a sample collection region may fill more effectively when it includes a back portion because the back portion may allow a flow of fluid (e.g., blood) flowing from the channel into the sample collection region to “double back.” The flow that doubles back may include flow through the sample collection region in a direction that includes a component that is opposite to the direction in which the fluid flowed through the channel and into the sample collection region. Such flow may happen in relatively desirable amounts within the back portion. However, it should be understood that it is also possible for at least some of the flow in the back portion to be flow in a direction that does not double back.

In some embodiments, like the embodiment shown in FIG. 8A, a sample collection region comprising a back portion further comprises a front portion. The front portion may include the locations in the sample collection region that do not make up any back portions present therein. The front portion may be closer to the channel terminus than to any portions of the channel upstream from the channel terminus. In FIG. 8A, the front portion of sample collection region 311 is indicated by reference sign 395. Fluid may flow into the front portion in a manner that primarily does not double back. However, it should be understood that it is also possible for at least some of the flow in the front portion to be flow in a direction that does double back.

FIGS. 8B and 8C illustrate a further non-limiting example of a sample collection region comprising front portion 395 and back portions 397 and 398 and the fluid flow that may occur therein. FIGS. 8B and 8C further include a dashed line 381 that shows the boundary between the front portion and the back portion. It should be understood that the dashed line does not correspond to any structural feature present in the sample collection region but merely demarcates the boundary between the regions therein.

As shown in FIG. 8C, in some embodiments, fluid (e.g., plasma) leaving channel terminus 317 travels radially outwards from channel terminus 317, such that fluid traveling through front portion 395 continues to travel at least partially in the same direction that it flowed through the channel. In contrast, fluid in back portions 397 and 398 may double back, at least partially reversing course and including a component that is opposite to the direction in which the fluid flowed through the channel and into the sample collection region. The flow in the back portions 397 and 398 may travel towards filter reception region 309, opposite the direction of fluid flow through the channel. In FIG. 8C, fluid flow 389 (indicated by a white arrow) passes through the channel has a direction defined by the channel. Once it exits the channel, fluid in the front portion of the sample collection region may to travel in directions 387 that at least partially align with the direction of fluid flow 389. Fluid flows 385 in the back portions of the sample collection region may double back.

Any of a variety of appropriate fluid volumes may be transported to the back portion of the sample collection region from the channel. In some embodiments, greater than or equal to 1%, greater than or equal to 2%, greater than or equal to 3%, greater than or equal to 5%, greater than or equal to 8%, greater than or equal to 10%, greater than or equal to 12%, greater than or equal to 15%, greater than or equal to 18%, greater than or equal to 20%, greater than or equal to 22%, greater than or equal to 25%, greater than or equal to 28%, greater than or equal to 30%, or greater than or equal to 32% of the plasma transported to the sample collection region is transported to the back portion. In some embodiments, less than or equal to 35%, less than or equal to 32%, less than or equal to 30%, less than or equal to 28%, less than or equal to 25%, less than or equal to 22%, less than or equal to 20%, less than or equal to 18%, less than or equal to 15%, less than or equal to 12%, less than or equal to 10%, less than or equal to 8%, less than or equal to 5%, less than or equal to 3%, or less than or equal to 2%, of the plasma transported to the sample collection region is transported to the back portion. Combinations of these ranges are also possible (e.g., greater than or equal to 1% and less than or equal to 35%, greater than or equal to 2% and less than or equal to 30%, or greater than or equal to 5% and less than or equal to 25%). Other ranges are also possible. When a sample collection region comprises two or more back portions, each back portion may independently receive an amount of the plasma in one or more of the above-referenced ranges and/or all of the back portions together may receive an amount of the plasma in one or more of the above-referenced ranges

In some embodiments, the sample collection region is laterally bounded by a boundary and/or a terminus of a channel. For example, the sample collection region may be bounded by both the boundary and the lateral terminus of the channel. In FIG. 3, sample collection region 311 is bounded by lateral terminus 317 of channel 315, and by boundary 319.

The boundary may comprise a section having a distance from a lateral terminus of the channel. For example, referring again to FIG. 3, boundary 319 comprises section 321 (indicated by the offset dashed line) having distance 323 from lateral terminus 317 of channel 315. In the context of the present disclosure, it has been inventively recognized that sample collection regions having boundaries comprising sections with relatively uniform distances from a lateral terminus of a channel may provide several advantages for the flow of fluid into the sample collection region. For example, sample collection regions with boundaries having a relatively uniform distance from a lateral terminus of a channel may uniformly draw fluid through the channel, and/or may be associated with desirable flow rates of fluids through the article. Without wishing to be bound by theory, the rate of fluid flow can be an important parameter when filtering plasma from blood cells, because although rapid flow is preferred for expedience, high flow rates may disadvantageously lyse blood cells and/or contaminate plasma with cellular material. Here, it has been recognized that sample collection regions with boundaries having a relatively uniform distance from a lateral terminus of a channel may produce relatively rapid fluid flow without substantial contamination of plasma. For this reason, in some embodiments, sample collection regions having a relatively uniform distance between a terminus of the channel and a portion of the boundary of the sample collection region, as illustrated in FIGS. 3-5, may offer advantages over sample collection regions having other forms.

In some embodiments, an average distance between the boundary section and the terminus of the channel is greater than or equal to 500 microns, greater than or equal to 750 microns, greater than or equal to 1000 microns, greater than or equal to 1250 microns, greater than or equal to 1500 microns, greater than or equal to 2 mm, greater than or equal to 3 mm, greater than or equal to 4 mm, greater than or equal to 5 mm, greater than or equal to 6 mm, greater than or equal to 7 mm, or greater. In some embodiments, average distance between the boundary section and the terminus of the channel is less than or equal to 10 mm, less than or equal to 9 mm, less than or equal 8 mm, less than or equal to 7 mm, less than or equal to 6 mm, less than or equal to 5 mm, less than or equal to 4 mm, less than or equal to 3 mm, less than or equal to 2 mm, less than or equal to 1500 microns, less than or equal to 1250 microns, less than or equal to 1000 microns, less than or equal to 750 microns, or less. Combinations of these ranges are possible (e.g., greater than or equal to 500 microns and less than or equal to 10 mm, greater than or equal to 500 microns and less than or equal to 1500 microns, greater than or equal to 750 microns and less than or equal to 1250 microns, or greater than or equal to 4 mm and less than or equal to 8 mm). Other ranges are also possible.

The distance between the section and the terminus of the channel may be exactly uniform in some embodiments. In other embodiments, the distance between the section and the terminus of the channel may vary. For example, if the sample collection region has the form of a sector of a circle that is centered on a point other than the channel terminus, the distance between the section and the channel terminus will be non-uniform. As another example, the section may be a section of a boundary of a polygonal sample collection region, or of a sample collection region that has a distortion, e.g., resulting from a cut, gap, perforation, or boundary feature. In some embodiments, this variation may be relatively small, which may advantageously allow the article to exhibit many of the same properties as a distance that is exactly uniform. According to some embodiments, a standard deviation of a distance from the terminus of the channel to the section is greater than or equal to 0%, greater than or equal to 1%, greater than or equal to 5%, greater than or equal to 10%, greater than or equal to 15%, greater than or equal to 20%, or greater. In some embodiments, standard deviation of a distance from the terminus of the channel to the section is less than or equal to 50%, less than or equal to 40%, less than or equal to 30%, less than or equal to 25%, less than or equal to 20%, less than or equal to 15%, less than or equal to 10%, less than or equal to 5%, or less. Combinations of these ranges are possible (e.g., greater than or equal to 0% and less than or equal to 50%, greater than or equal to 0% and less than or equal to 30%, or greater than or equal to 1% and less than or equal to 25%). Other ranges are also possible.

In some embodiments, the section makes up greater than or equal to 15%, greater than or equal to 20%, greater than or equal to 25%, greater than or equal to 30%, greater than or equal to 40%, greater than or equal to 50%, greater than or equal to 60%, greater than or equal to 70%, greater than or equal to 80%, greater than or equal to 90%, or more of the boundary. In some embodiments, the section makes up less than or equal to 99%, less than or equal to 95%, less than or equal to 90%, less than or equal to 80%, less than or equal to 70%, less than or equal to 60%, or less of the boundary. Combinations of these ranges are possible (e.g., greater than or equal to 15% and less than or equal to 99%, or greater than or equal to 50% and less than or equal to 90%). Other ranges are also possible. The section may be continuous or may comprise two or more parts that are discontinuous.

In some embodiments, the boundary of the sample collection region intersects a channel boundary at an angle and/or includes a bend. For example, referring to FIG. 9, the boundary of sample collection region 311 intersects the boundary of channel 315 at angle 361 and the boundary of sample collection region 311 undergoes a bend at angle 363. As can be seen in FIG. 9, the magnitudes of the angles at which the boundary of the sample collection region intersects the channel may be parametrized by the angular range they span that is internal to the sample collection region and the channel. Similarly, the magnitudes of the angles at which the boundary of the sample collection region undergoes a bend may be parametrized by the angular range they span that is internal to the sample collection region. When parametrized in these manners, these angles may be referred to as “interior angles.” Of course, angles such as 361 and 363 could be eliminated by rounding the corners of the sample collection region boundary with those angles, and a sample collection region boundary may lack intersections at angles and/or bends.

The existence of a back portion may result from the existence of a relatively large interior angle between a boundary of the channel and a boundary of the sample collection region, as described above. Thus, in some embodiments, it may be advantageous for a boundary of the channel to intersect a boundary of the sample collection region at an interior angle having a relatively large magnitude and/or for a sample collection region to include an interior angle having a relatively large magnitude. However, an interior angle between the boundary of the channel and the boundary of the sample collection region is not required. For example, referring again to FIG. 9, interior angle 361 could be, in some embodiments, replaced by a rounded corner that does not form a large interior angle, and would still include back portions 397 and 398.

In some embodiments, a boundary of the sample collection region intersects a boundary of the channel at an interior angle and/or comprises an interior angle of greater than or equal to 180°, greater than or equal to 190°, greater than or equal to 200°, greater than or equal to 210°, greater than or equal to 220°, greater than or equal to 230°, greater than or equal to 240°, greater than or equal to 250°, greater than or equal to 260°, greater than or equal to 270°, greater than or equal to 280°, greater than or equal to 290°, greater than or equal to 300°, greater than or equal to 310°, or greater than or equal to 320°. In some embodiments, a boundary of the sample collection region intersects a boundary of the channel at an interior angle and/or comprises an interior angle of less than or equal to 330°, less than or equal to 320°, less than or equal to 310°, less than or equal to 300°, less than or equal to 290°, less than or equal to 280°, less than or equal to 270°, less than or equal to 260°, less than or equal to 250°, less than or equal to 240°, less than or equal to 230°, less than or equal to 220°, less than or equal to 210°, less than or equal to 200°, or less than or equal to 190°. Combinations of these ranges are also possible (e.g., greater than or equal to 1800 and less than or equal to 330°, greater than or equal to 1900 and less than or equal to 300°, or greater than or equal to 2500 and less than or equal to 290°). Other ranges are also possible.

In embodiments in which a sample collection region comprises a front portion and a back portion, it may be useful to characterize the boundary of the sample collection region as including front and back boundary portions. For example, in some embodiments, a back portion of the sample collection region is at least partially bounded by a back boundary portion. Like the back portion, the back boundary portion may be closer to a portion of the channel directly upstream from the channel terminus than it is from the channel terminus itself. For example, referring again to FIG. 8A, point 375 positioned on the back boundary portion is closer to point 377 of the channel directly upstream from channel terminus 317 than it is to point 379 at the channel terminus (compare the lengths of lines 371 and 373). Thus, according to some embodiments, point 375 is part of the back boundary of back portion 398. Likewise, a front portion such as front portion 395 may be at least partially bounded by a front boundary portion that is closer to the channel terminus than to any other portion of the channel. A boundary of a sample collection region may comprise more than one back boundary portion and/or more than one front boundary portion (e.g., two or more back boundary portions that are separated by a front boundary portions). The back boundary portion(s) and the front boundary portion(s) may have a total length equaling a length of the boundary of the sample collection region, exclusive of the channel terminus, according to some embodiments.

Any of a variety of suitable proportions of the boundary of the sample collection region may be back boundary portions. In some embodiments, a back boundary portion of the sample collection region makes up greater than or equal to 1%, greater than or equal to 2%, greater than or equal to 3%, greater than or equal to 5%, greater than or equal to 8%, greater than or equal to 10%, greater than or equal to 12%, greater than or equal to 15%, greater than or equal to 18%, greater than or equal to 20%, greater than or equal to 22%, greater than or equal to 25%, greater than or equal to 28%, greater than or equal to 30%, or greater than or equal to 32% of the length of the boundary of the sample collection region. In some embodiments, a back portion of the sample collection region makes up less than or equal to 35%, less than or equal to 32%, less than or equal to 30%, less than or equal to 28%, less than or equal to 25%, less than or equal to 22%, less than or equal to 20%, less than or equal to 18%, less than or equal to 15%, less than or equal to 12%, less than or equal to 10%, less than or equal to 8%, less than or equal to 5%, less than or equal to 3%, or less than or equal to 2% of the length of the boundary of the sample collection region. Combinations of these ranges are also possible (e.g., greater than or equal to 1% and less than or equal to 35%, greater than or equal to 2% and less than or equal to 30%, or greater than or equal to 5% and less than or equal to 25%). Other ranges are also possible. It should be understood that the aforementioned ranges may refer to the proportion of the boundary of the sample collection region occupied by a single back portion, or to a proportion of the boundary of the sample collection region occupied collectively by back portions, as the disclosure is not so limited.

In some embodiments, it may be possible to remove one or more samples from an article described herein. The sample(s) and sample collection region(s) may be removed from the article together (e.g., by way of a biopsy punch, by way of peeling), or the sample(s) may be removed from the article without also removing the sample collection region(s). In some embodiments, the sample collection region is configured to be removed from the article. For example, the absorbent layer may comprise cuts, gaps, or perforations surrounding the sample collection region that advantageously facilitate removal of the sample collection region. The absorbent layer may comprise boundary features such as tabs, loops, or holes to facilitate removal of the sample collection region. In some embodiments, the sample collection region is configured to be removed using tweezers.

As described above, articles described herein may comprise a channel. In some embodiments, a layer comprises a channel. For example, a channel may be present in the absorbent layer. For example, in FIGS. 1A-1B, channel 115 is present in absorbent layer 105, and in FIG. 2, channel 215 is present in absorbent layer 205. The channel may have any of a variety of suitable dimensions. In some embodiments, the channel extends through the thickness of the layer. In other words, some channels may have the same thickness as the layers in which they are positioned. Some channels may span a distance less than the width and/or length of the layer. In some embodiments, one or more channels may have dimensions that aid in metering of a fluid sample. The channel(s) may have a volume, dimension, and/or shape that promotes flow of a desired volume of the fluid sample therein and/or therethrough.

In some embodiments, a channel may fluidically connect portions of an article. For instance, a channel may connect a sample collection region to a filter reception region (as described in greater detail below). In some embodiments, a channel fluidically connects a filter (e.g., a first filter, a second filter) to a sample collection region. Two article portions (e.g., filters, layers, regions of layers) may be fluidically connected if, in at least some configurations of the article, a fluid (e.g., a blood sample) may pass between them. Thus, in some embodiments, an article is configured such that fluid may be transmitted through the channel. For example, in some embodiments the absorbent layer is configured to transport fluid to the sample collection region via the channel.

An article may comprise a channel (e.g., a channel connecting a filter or filter reception region with a sample collection region) with a thickness or height of greater than or equal to 50 microns, greater than or equal to 75 microns, greater than or equal to 100 microns, greater than or equal to 125 microns, greater than or equal to 150 microns, greater than or equal to 200 microns, greater than or equal to 250 microns, greater than or equal to 300 microns, greater than or equal to 400 microns, greater than or equal to 500 microns, or greater than or equal to 750 microns. The article may comprise a channel with a thickness or height of less than or equal to 1000 microns, less than or equal to 750 microns, less than or equal to 500 microns, less than or equal to 400 microns, less than or equal to 300 microns, less than or equal to 250 microns, less than or equal to 200 microns, less than or equal to 150 microns, less than or equal to 125 microns, or less than or equal to 100 microns. Combinations of the above-referenced ranges are also possible (e.g., greater than or equal to 50 microns and less than or equal to 1000 microns, greater than or equal to 50 microns and less than or equal to 500 microns, or greater than or equal to 50 microns and less than or equal to 100 microns). Other ranges are also possible.

Channels (e.g., connecting a filter or filter reception region with a sample collection region) in articles may have any of a variety of suitable widths. In some embodiments, an article comprises a channel with a width of greater than or equal to 0.01 cm, greater than or equal to 0.02 cm, greater than or equal to 0.05 cm, greater than or equal to 0.1 cm, greater than or equal to 0.2 cm, greater than or equal to 0.5 cm, greater than or equal to 1 cm, greater than or equal to 1.5 cm, greater than or equal to 2 cm, or greater. The article may comprise a channel with a width of less than or equal to 5 cm, less than or equal to 3 cm, less than or equal to 2 cm, less than or equal to 1.5 cm, less than or equal to 1 cm, less than or equal to 0.5 cm or less. Combinations of the above-referenced ranges are also possible (e.g., greater than or equal to 0.01 cm and less than or equal to 5 cm, greater than or equal to 0.01 cm and less than or equal to 2 cm, greater than or equal to 0.2 cm and less than or equal to 5 cm, or greater than or equal to 1.5 cm and less than or equal to 3 cm). Other ranges are also possible.

Channels (e.g., connecting a filter or filter reception region with a sample collection region) in articles may have any of a variety of suitable lengths. In some embodiments, an article comprises a channel with a length of greater than or equal to 0.01 cm, greater than or equal to 0.02 cm, greater than or equal to 0.05 cm, greater than or equal to 0.1 cm, greater than or equal to 0.2 cm, greater than or equal to 0.5 cm, greater than or equal to 1 cm, greater than or equal to 1.5 cm, greater than or equal to 2 cm, or greater. The article may comprise a channel with a length of less than or equal to 5 cm, less than or equal to 3 cm, less than or equal to 2 cm, less than or equal to 1.5 cm, less than or equal to 1 cm, less than or equal to 0.5 cm or less. Combinations of the above-referenced ranges are also possible (e.g., greater than or equal to 0.01 cm and less than or equal to 5 cm, greater than or equal to 0.01 cm and less than or equal to 2 cm, greater than or equal to 0.2 cm and less than or equal to 5 cm, or greater than or equal to 1.5 cm and less than or equal to 3 cm). Other ranges are also possible.

Channels (e.g., channels connecting a filter or filter reception region with a sample collection region) in articles may have any of a variety of suitable aspect ratios (i.e., ratios of the channel length to the channel width). In some embodiments, an article comprises a channel with an aspect ratio of greater than or equal to 0.5, greater than or equal to 1, greater than or equal to 1.2, greater than or equal to 1.5, greater than or equal to 2, greater than or equal to 3, or greater. The article may comprise a channel with an aspect ratio of less than or equal to 5, less than or equal to 3, less than or equal to 2, less than or equal to 1.5, less than or equal to 1.2, or less. Combinations of the above-referenced ranges are also possible (e.g., greater than or equal to 0.5 and less than or equal to 5). Other ranges are also possible.

In some embodiments, an article comprises a channel with a volume of greater than or equal to 1 microliter, greater than or equal to 2 microliters, greater than or equal to 5 microliters, greater than or equal to 10 microliters, greater than or equal to 15 microliters, greater than or equal to 20 microliters, greater than or equal to 30 microliters, greater than or equal to 40 microliters, greater than or equal to 50 microliters, greater than or equal to 75 microliters, greater than or equal to 100 microliters, greater than or equal to 150 microliters, greater than or equal to 200 microliters, greater than or equal to 300 microliters, greater than or equal to 400 microliters, greater than or equal to 500 microliters, or greater than or equal to 750 microliters. The article may comprise a channel with a volume of less than or equal to 1 mL, less than or equal to 750 microliters, less than or equal to 500 microliters, less than or equal to 400 microliters, less than or equal to 300 microliters, less than or equal to 200 microliters, less than or equal to 150 microliters, less than or equal to 100 microliters, less than or equal to 75 microliters, less than or equal to 50 microliters, less than or equal to 40 microliters, less than or equal to 30 microliters, less than or equal to 20 microliters, less than or equal to 15 microliters, less than or equal to 10 microliters, less than or equal to 5 microliters, or less than or equal to 2 microliters. Combinations of the above-referenced ranges are also possible (e.g., greater than or equal to 1 microliter and less than or equal to 1 mL, greater than or equal to 1 microliter and less than or equal to 50 microliters, or greater than or equal to 100 microliters and less than or equal to 300 microliters). Other ranges are also possible.

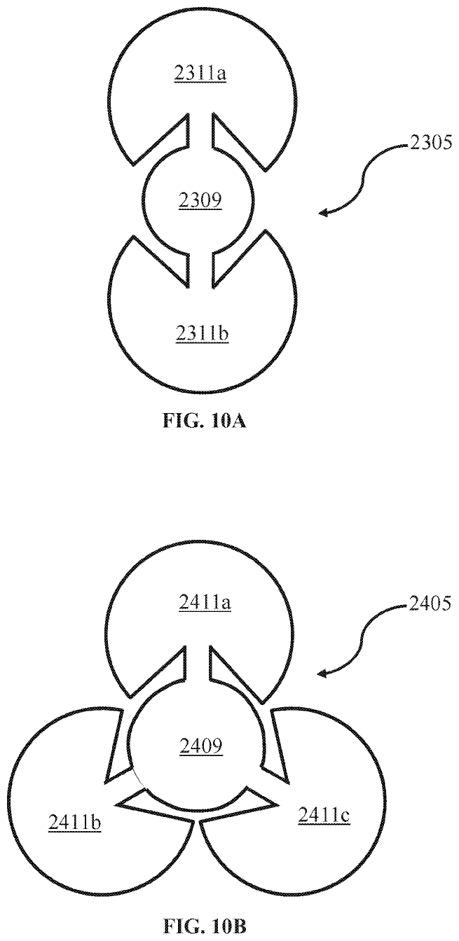

In some embodiments, it may be advantageous for an article to partition a fluid sample (e.g., plasma) into a plurality of sample collection regions, rather than into a single sample collection region. Thus, in some embodiments the article comprises a plurality of sample collection regions (e.g., 2, 3, 4, 5, 6, or more sample collection regions), which may be fluidically connected to a common filter reception region by channels. FIG. 10A presents such an embodiment, wherein an absorbent layer 2305 comprises a first sample collection region 2311a and a second sample collection region 2311b, each fluidically connected to filter reception region 2309 by a channel. FIG. 10B presents another such embodiment, wherein an absorbent layer 2405 comprises a first sample collection region 2411a, a second sample collection region 2411b, and a third sample collection region 2411c, each connected to a filter reception region 2409. A fluid (e.g., plasma) may be transported through the filter such that, in some embodiments, a first a first portion of the fluid is transported in a first lateral direction within the absorbent layer to the first sample collection region and a second portion of the fluid is transported in a second lateral direction non-parallel to the first lateral direction to a second sample collection region. For example, in FIG. 10A, a first portion of a fluid transported to filter reception region 2309 is transported in a first lateral direction from filter reception region 2309 to first sample collection region 2311a and a second portion of plasma transported to filter reception region 2309 is transported in a second lateral direction from filter reception region 2309 to second sample collection region 2311b. The first portion of plasma and the second portion of plasma may have the same volume. Without wishing to be bound by any particular theory, the transport of plasma portions with identical volumes may occur, for example, if the sample collection regions have sufficient symmetry, as discussed below. Of course, in some embodiments, the first plasma portion and the second plasma portion have different volumes.

The properties of sample collection regions described herein may characterize some or all of the sample collection regions in articles comprising multiple sample collection regions. For example, in some embodiments, some or all of the sample collection regions are laterally spaced from a filter, are fluidically connected to the filter, and/or are configured to receive plasma from which blood cells have been separated. The laterally spaced sample collection regions may each be fluidically connected to a filter (or filters) via a channel having a terminus that forms a portion of the boundary of the sample collection region.

In some embodiments, a plurality of sample collection regions and/or a plurality of channels may be uniform in one or more ways and/or may be arranged to have symmetry around a filter and/or a filter reception region. For example, in some embodiments, the plurality of sample collection regions have the same area and/or a same volume. For example, sample collection regions 2311a and 2311b of FIG. 10A have the same volume and the same area. As another example, in some embodiments, the sample collection regions are situated at regular angles around the filter. For example, sample collection regions 2311a and 2311b of FIG. 10A are situated at regular, 180° angles around filter reception region 2309 (situated beneath a filter, unshown), and sample collection regions 2411a, 2411b, and 2411c of FIG. 10B are situated at regular, 120° angles around filter reception region 2409 (situated beneath the filter, unshown). In some such embodiments, the sample collection region has rotational symmetry around a center point overlapping the filter. As another example, in some embodiments, the sample collection regions are situated at a same distance from the filter. For example, as shown in FIGS. 10A-10B, the sample collection regions may be separated from a filter reception region by channels of the same length. Regular angles, regular distances from the filter, and/or regular volumes of the sample collection region may each individually be associated with the uniform distribution of fluid flow between the sample collection regions, which may be beneficial for collection of uniform volumes of plasma in each sample collection region.

Of course, symmetry is not required, and in some embodiments it may be advantageous to avoid symmetry of the sample collection regions, e.g., by using sample collection regions of different areas and/or different volumes. In some embodiments, sample collection regions of different sizes may be used to partition the fluid (e.g., the plasma) entering the absorbent layers into different volumes. For example, in some embodiments a first sample collection region is configured to receive a first volume of plasma greater than a second volume of plasma which a second sample collection region is configured to receive from the same filter.

Generally, the use of multiple sample collection regions may be advantageous, e.g., for the performance of simultaneous assays on the same plasma sample. For example, plasma collected in a first sample collection region may be used for a first assay and plasma collected in a second sample collection region may be used for a second assay. In some embodiments, different assays require different plasma volumes, and asymmetry of the sample collection regions may be useful for preparing plasma samples of different volumes for different assays.

It is also possible for plasma from multiple sample collection regions to be pooled. In other words, multiple sample collection regions may be collected and employed together for a single purpose (e.g., a single assay). This may be an advantageous alternative to using multiple sample regions with different volumes when performing multiple assays with different plasma requirements. In some embodiments, pooling sample collection regions introduces the benefits of symmetry for fluid flow through the article, while simultaneously permitting the use of asymmetric sample volumes for assaying samples. According to some embodiments, plasma from all the sample collection regions may be pooled. Pooling of plasma from all sample collection regions may allow recovery of a large portion of the plasma filtered by the device for a demanding assay without requiring use of a separate article comprising only a single sample collection region.

Elsewhere herein, the use of articles comprising multiple filters, filter reception regions, and sample collection regions as fluidically isolated components of the same article is described. It should, of course, be understood that an article can comprise multiple, fluidically isolated sets of filters and absorption layers, wherein each absorption layer comprises multiple sample collection regions fluidically connected to each filter.

It can be advantageous, when collecting plasma for an assay, to simultaneously collect a whole blood sample. The collection of whole blood may be useful if whole blood or cellular assays are required, and it may be advantageous for measurement consistency to know that a whole blood sample originated from the same sample as a filtered plasma sample. Thus, according to some embodiments, the disclosure is directed towards articles comprising whole blood collection regions in addition to sample collection regions.

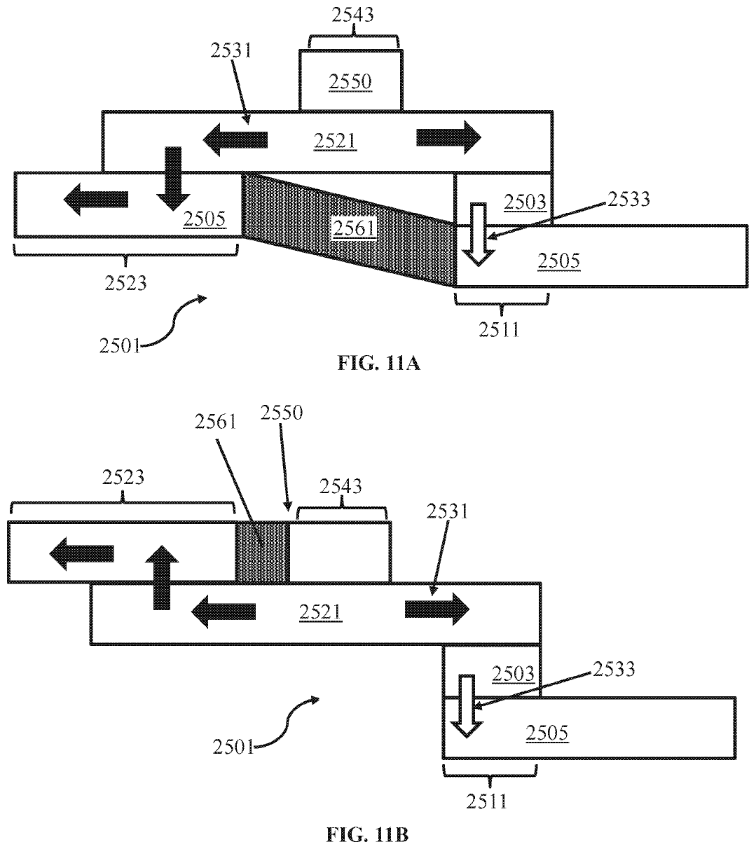

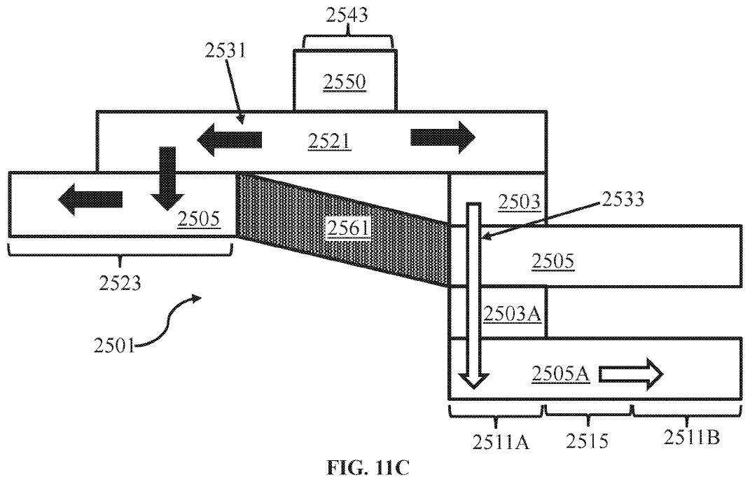

FIG. 11A presents a non-limiting, cross-sectional schematic illustration of such an article, article 2501. Article 2501 comprises first layer 2550 (which may be an absorbent layer), comprising sample inlet 2543. A blood sample comprising blood cells and plasma may be passed through inlet 2543 to fluid distribution layer 2521 disposed beneath sample inlet 2543. Fluid distribution layer 2521 may directly contact first layer 2550, as shown in FIG. 11A, or may be separated by one or more intervening layers. As indicated by dark arrows 2531 of FIG. 11A, blood may spread through the fluid distribution layer, such that a portion of the blood is passed to a whole blood collection region 2523 (a type of sample collection region), and another portion of blood is passed to a filter 2503. The filter may separate the blood cells from the plasma and transport the plasma to an absorbent layer 2505 comprising a plasma collection region 2511 (a type of sample collection region). White arrows 2533 represent the flow of plasma from filter 2503 and towards plasma collection region 2511.

As shown in FIG. 11A, whole blood collection region 2523 may be part of an absorbent layer 2505 that also comprises plasma collection region 2511 but is fluidically isolated from plasma collection region 2511 (e.g., by a region 2561 that has been rendered impermeable to fluids, and/or that bounds the whole blood collection region and the plasma collection region). Of course, it should be understood that “fluidically isolated” as used herein refers to the fluidic isolation of the separate regions within the layer, in some embodiments, since whole blood collection region 2523 and plasma collection region 2511 are fluidically connected by a fluidic pathway passing through filter 2503.

Alternative arrangements of the whole blood collection region are also possible. For example, the whole blood correction region may be part of the first layer and may be fluidically isolated from the inlet. FIG. 11B provides such an example, wherein first layer 2550 of article 2501 comprises inlet 2543, isolated from whole blood collection region 2523 by impermeable barrier 2561 of first layer 2550. As with article 2501 of FIG. 11A, blood may be passed through inlet 2543 to fluid distribution layer 2521. Dark arrows 2531 represent the flow of blood through the article, and illustrate that a first portion of the blood can be passed from fluid distribution layer 2521 to whole blood collection region 2523 of first layer 2550, while a second portion of the blood can be transported to absorbent layer 2505 through filter 2503 and to plasma collection region 2511.

As shown in FIG. 11A and FIG. 11B, the whole blood collection region may be configured to receive blood directly from the fluid distribution layer 2521 of the first layer without filtering the blood. Thus, the whole blood collection region collects whole blood, in some embodiments. According to some embodiments, the whole blood collection region permits lateral flow of blood within the absorbent layer, as shown in FIGS. 11A-11B. However, it should of course be understood that the whole blood collection region may also directly overlap the fluid distribution layer, such that lateral flow of the blood is minimal. The whole blood collection region may be configured (e.g., sized and/or treated) to retain any of a variety of appropriate blood volumes. For example, the whole blood collection region may be configured to store a blood volume similar to the plasma volume stored by the plasma collection region, in some embodiments. Like the plasma collection region, the whole blood collection region may be treated with any of a variety of suitable reagents, described in greater detail below.

The whole blood collection region may be configured to receive blood directly from the fluid distribution layer, or may be separated from the fluid distribution layer by one or more intervening layers, as long as the intervening layers are not filters capable of filtering blood cells from plasma.

The whole blood collection region may have any of a variety of suitable geometries. For example, in some embodiments the whole blood collection region is separated (e.g., laterally) from the fluid distribution layer by a channel. In some embodiments, the whole blood collection region is bounded by a boundary and a channel terminus. According to some embodiments, the whole blood collection region has a same area as a plasma collection region. In some embodiments, the whole blood collection region has a same volume as a plasma collection region. An article may comprise multiple plasma collection regions connected to a same filter as discussed above, and may also comprise a whole blood collection region and a fluid distribution layer of a suitable geometry and volume.

In some embodiments, articles suitable for collecting both whole blood and plasma comprise one or more further layers. As one example, in some embodiments, an article comprises an additional absorbent layer disposed beneath the absorbent layer comprising the plasma collection region (and, optionally, the whole blood collection region). As another example, in some embodiments, a second filter is disposed in between these two absorbent layers. One example of an article having this design is shown in FIG. 11C. As shown in FIG. 11C, second filter 2503A is disposed beneath the plasma collection region in absorbent layer 2505 and second absorbent layer 2505A is disposed beneath second filter 2503A. After flowing through the plasma collection region, the blood can flow through the second filter and into the second absorbent layer. This absorbent layer may itself comprise a plasma collection region (shown in FIG. 11C by reference sign 2511A). Accordingly, the plasma collection region in the second absorbent layer may be configured to receive fluid from the second filter.

In some embodiments, a second absorbent layer further comprises one or more additional components.

As one example, in some embodiments, a second absorbent layer may comprise yet another plasma collection region. This plasma collection region may be laterally spaced from the other plasma collection region in the layer and in fluidic communication therewith via a channel. This is shown schematically in FIG. 11C, in which plasma collection region 2511A in second absorbent layer 2505A is laterally spaced from and in fluidic communication with plasma collection region 2511B via channel 2515.

As another example, in some embodiments, a second absorbent layer further comprises a whole blood collection region. When present, the whole blood collection region in the second absorbent layer may be configured to receive fluid directly from the first absorbent layer (e.g., from a whole blood collection region therein). It is also possible for the whole blood collection region in the second absorbent layer to receive fluid indirectly from the first absorbent layer (e.g., from a whole blood collection region therein) so long as it does not pass through a filter that would remove components thereof. When both a whole blood collection region and a plasma collection region are present in a second absorbent layer, they may be fluidically isolated from each other in the second absorbent layer.

FIG. 11D schematically depicts an article comprising a second absorbent layer that further comprises a whole blood collection region. In FIG. 11D, the second absorbent layer 2505A comprises plasma collection regions 2511A and 2511B that are fluidically isolated from a whole blood collection region 2523A by a region 2561A. Region 2561A is a region that has been rendered impermeable to fluids and/or that bounds the whole blood collection region and the plasma collection region.

As a third example, a second absorbent layer may comprise two whole blood collection regions. The whole blood collection regions may be laterally spaced from each other in the layer and in fluidic communication therewith via a channel. For instance, as shown in FIG. 11D, whole blood collection region 2523A in second absorbent layer 2505A is laterally spaced from and in fluidic communication with plasma collection region 2523B via channel 2515A.

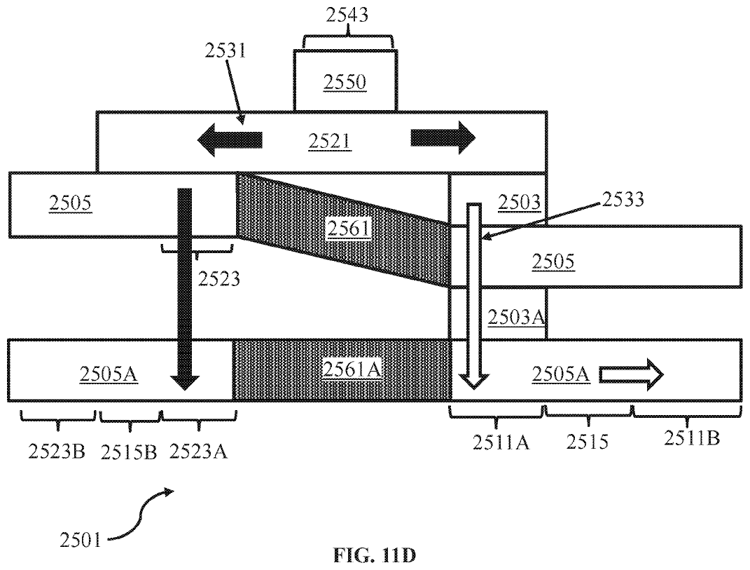

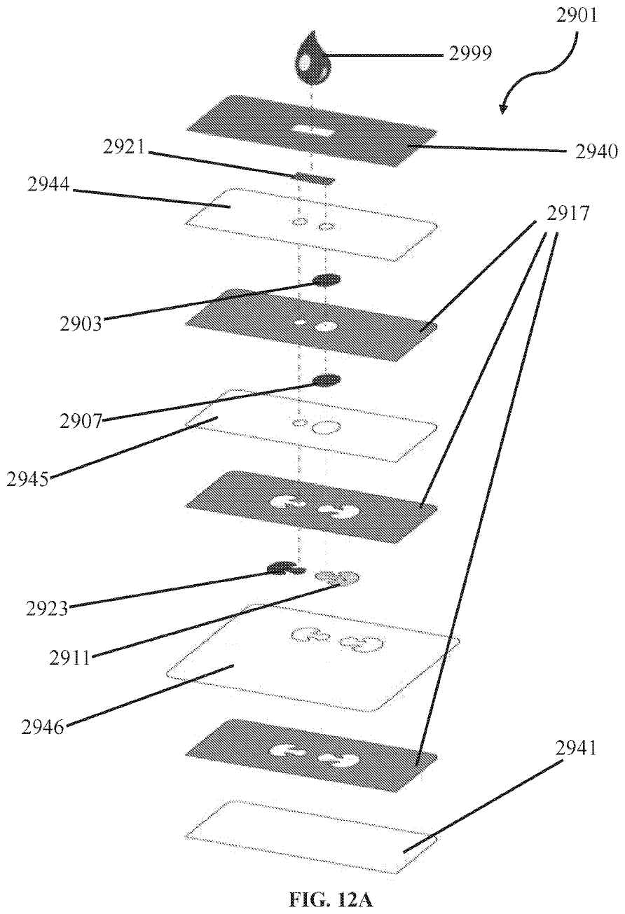

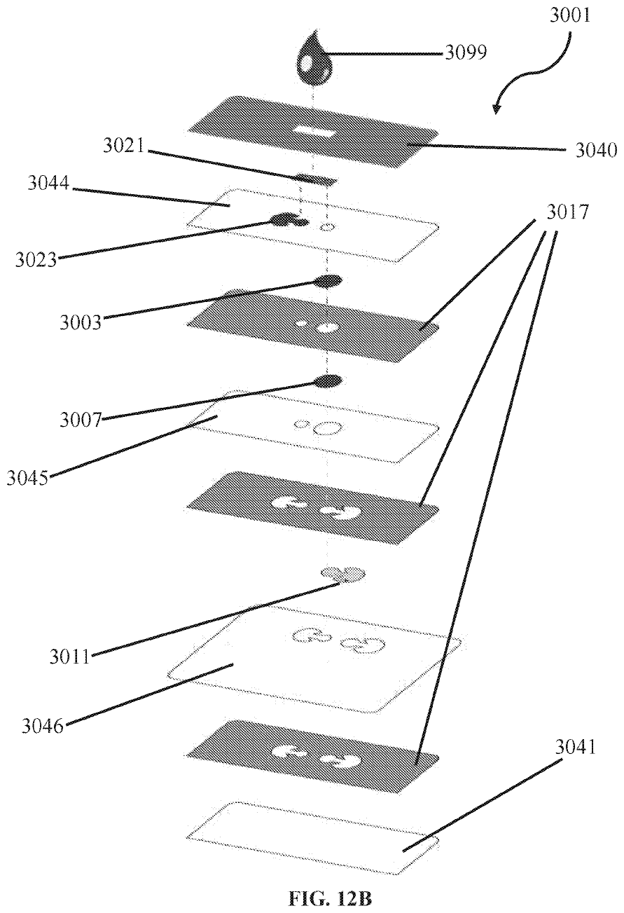

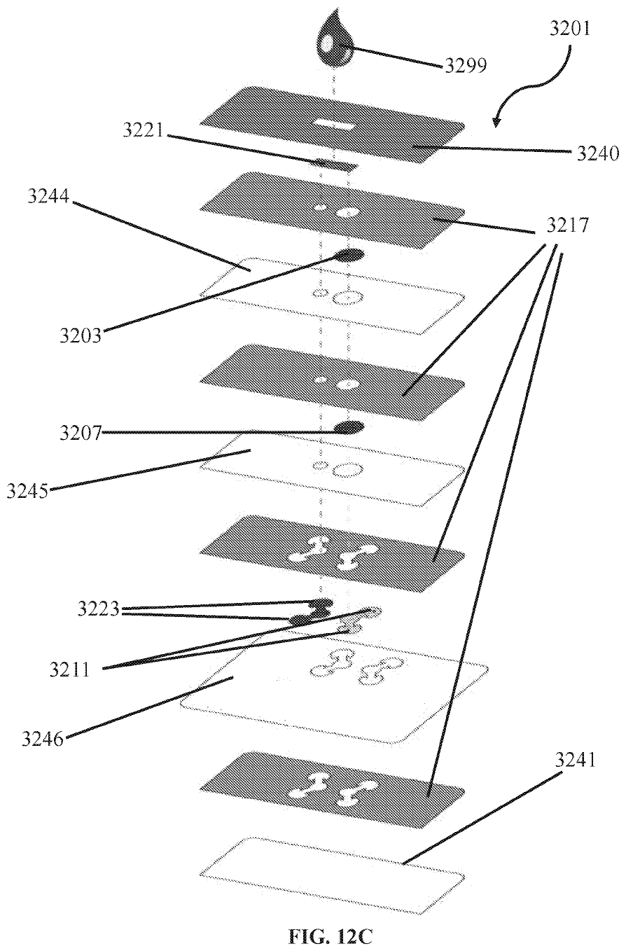

FIG. 12A presents an exploded-perspective schematic illustration of a non-limiting article 2901 for collecting both whole blood and plasma, according to some embodiments. The article comprises adhesive layer 2917, as well as a fluid distribution layer 2921 that is configured to pass a first portion of blood 2999 to whole blood collection region 2923 and to pass a second portion of blood 2999 through filters 2903 and 2907, to purify plasma that may be passed to sample collection region 2911. Also shown are portions 2940 and 2941 of the support structure, and layer housings 2944, 2945, and 2946, which are configured to maintain the relative positions of article layers.

FIG. 12B presents an exploded-perspective schematic illustration of a non-limiting article 3001, according to some embodiments, which is similar to article 2901 presented in FIG. 12A. However, as shown, article 3001 includes blood collection region 3023 in one absorbent layer that directly contacts fluid distribution layer 3021, and includes plasma collection region 3011 in another absorbent layer separated from fluid distribution layer 3021 by filters 3003 and 3007. As in article 2901, adhesive layers 3017 separate layers of the article. Also shown are portions 3040 and 3041 of the support structure, and layer housings 3044, 3045, and 3046, which are configured to maintain the relative positions of article layers.

FIG. 12C presents an exploded-perspective schematic illustration of a non-limiting article 3201, according to some embodiments, which is similar to article 2901 presented in FIG. 12A. However, as shown, article 3201 includes two blood collection regions 3223 in one absorbent layer that are configured to receive blood from fluid distribution layer 3221, and includes two plasma collection regions 3211 separated from fluid distribution layer 3221 by filters 3203 and 3207. As in article 2901, adhesive layers 3217 separate layers of the article. Also shown are portions 3240 and 3241 of the support structure, and layer housings 3244, 3245, and 3246, which are configured to maintain the relative positions of article layers.

In some embodiments, it may be desirable to recover a consistent sample volume, e.g., by filling a sample collection region to a target volume while removing any excess sample volume transported to the sample collection region. In some embodiments, the removal of excess sample volume from the sample collection region may be achieved using a second, absorbent layer. The second, absorbent layer may be a relatively thick, absorbent layer configured to wick and absorb any excess fluid from a first absorbent layer to which it is adjacent. In some embodiments, a second, absorbent layer may be positioned as the lowermost layer. This may be beneficial, for instance, in the case where a large amount of fluid is applied to the fluidic device. This large amount of fluid may cause an amount of fluid to flow to the sample collection regions that is larger than the amount desired for later analysis thereof. It is also possible for a second, absorbent layer to be positioned above a porous, absorbent layer comprising a sample collection region. A second, absorbent layer in fluidic communication with sample regions (e.g., positioned directly above or below the sample regions) may wick fluid from these sample regions to an extent such that the desired amount of fluid is retained therein.

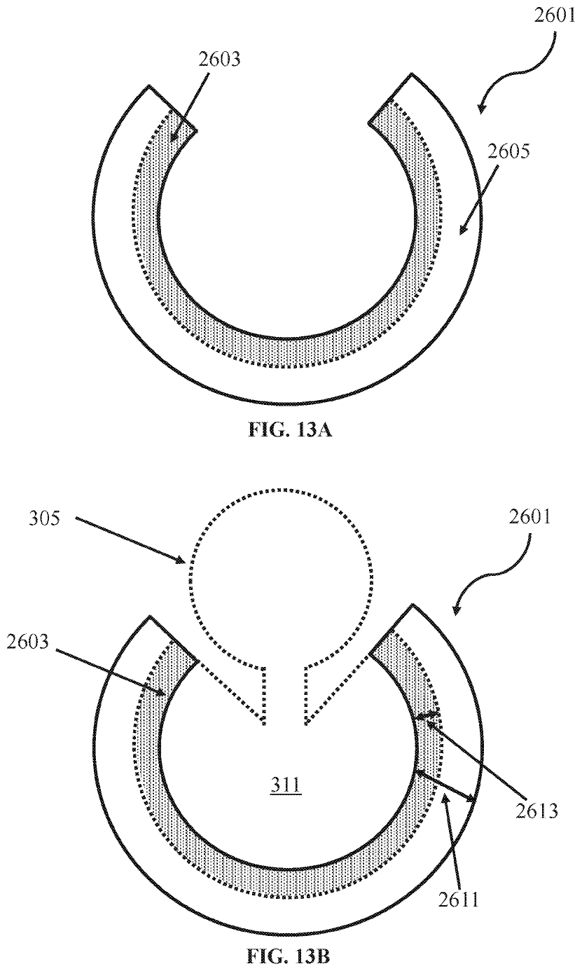

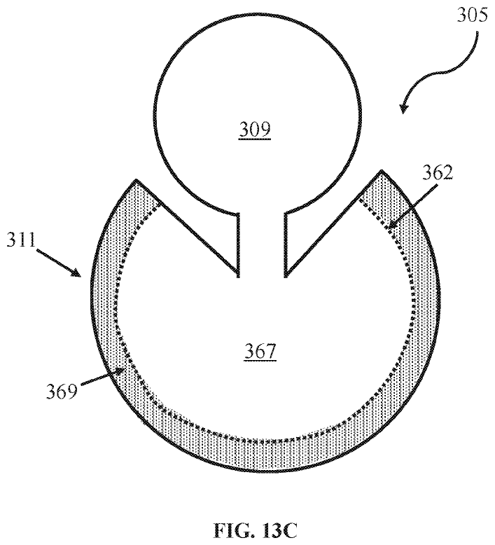

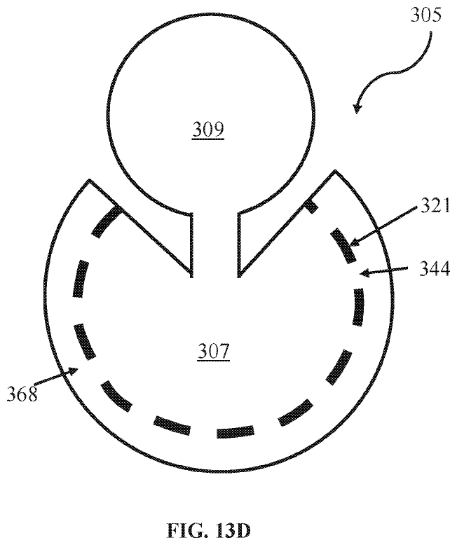

According to some embodiments, the second, absorbent layer contacts the sample collection region. For example, the second, absorbent layer may comprise an overflow region comprising a receiving portion that overlaps a portion of the sample collection region. FIG. 13A presents a schematic, top-view illustration of an overflow region 2601 of an absorbent layer, wherein overflow region 2601 is configured to overlap a sample collection region at receiving portion 2603 but does not overlap the sample collection portion at portion 2605 of overflow region 2601. Overflow region 2601 may be bounded at least partially in the plane of the absorbent layer by a fluid impermeable barrier, as indicated by the solid boundary line, or may extend to an edge of the absorbent layer. It is also possible for overflow region 2601 to extend in at least one direction to an outer edge of the absorbent layer, thus being at least partially unbounded in the absorbent layer. FIG. 13B visually overlays overflow portion 2601 with a region the region of first absorbent layer 305 (bounded by a dashed line) originally presented in FIG. 3.

As shown in FIG. 13B, receiving portion 2603 overlaps a portion of sample collection region 311, thereby defining an overlap portion of the sample collection region by the area of overlap between the two layers. FIG. 13C illustrates absorbent layer 305, showing overlap portion 369 of sample collection region 311, which terminates at dashed line 362 corresponding to the inner boundary of overflow region 2601 presented in FIGS. 13A-13B. In some embodiments, a sample collection region does not overlap the overflow portion across the entire sample collection region. For example, referring again to FIG. 13C, sample collection region 311 comprises non-overlap portion 367 of sample collection region 311. According to some embodiments, the terminus of the channel opens into the non-overlap portion, e.g., so that the channel does not transport material to the second, absorbent layer without first filling the sample collection region of the first, absorbent layer.

In some embodiments, excess plasma may be transported out of a sample collection region to a receiving portion. The plasma may then be transported laterally from the receiving portion, wicking into the rest of the overflow region. In some embodiments, it may be advantageous for the overflow region to extend symmetrically outwards from the receiving portion, as shown in FIG. 13A, where portion 2605 extends a constant distance outward from receiving portion 2603. Without wishing to be bound by any particular theory, symmetrical extension of the overflow region from the receiving portion may facilitate more uniform fluid flow into the overflow portion, according to some embodiments. Similarly, in some embodiments, the receiving portion extends symmetrically inwards from the boundary of the sample collection region, as is shown in FIG. 13B. Symmetric extension of the receiving portion inwards from the boundary may be advantageous when, for example, fluid emanates radially from a channel terminus into the sample collection region, and must travel a relatively constant radial distance to reach the boundary, as may be true of fluid entering sample collection region 311. Without wishing to be bound by any particular theory, the radial symmetry of the receiving portion of the second, absorbent layer may result in improved sample uniformity e.g., by permitting fluid to leave the sample collection region at substantially the same time as the other fluid that entered the sample collection region at the same time.

The sample overflow region may have any of a variety of suitable geometries. In some embodiments, a distance from an outer boundary of a bounded overflow region (corresponding to a boundary portion of the overflow region farthest from the channel terminus of the first, absorbent layer) to an inner boundary of the receiving portion (corresponding to a portion of the boundary of the receiving portion that is closest to a channel terminus of the first, absorbent layer) varies by a relatively small amount. For example, referring again to FIG. 13B, distance 2611 may vary by a relatively small amount. In some embodiments, a distance to an outer boundary of the overflow region from an inner boundary of the receiving portion varies by greater than or equal to 1%, greater than or equal to 2%, greater than or equal to 5%, greater than or equal to 8%, greater than or equal to 10%, greater than or equal to 12%, greater than or equal to 15%, greater than or equal to 18%, greater than or equal to 20%, greater than or equal to 22%, greater than or equal to 25%, greater than or equal to 30%, greater than or equal to 35%, greater than or equal to 40%, greater than or equal to 45%, greater than or equal to 50%, or greater than or equal to 60% of the average distance between the outer boundary of the overflow region and the inner boundary of the receiving portion. In some embodiments, a distance to an outer boundary of the overflow region from an inner boundary of the receiving portion varies by less than or equal to 70%, less than or equal to 60%, less than or equal to 50%, less than or equal to 45%, less than or equal to 40%, less than or equal to 35%, less than or equal to 30%, less than or equal to 25%, less than or equal to 22%, less than or equal to 20%, less than or equal to 18%, less than or equal to 15%, less than or equal to 12%, less than or equal to 10%, less than or equal to 8%, less than or equal to 5%, or less than or equal to 2% of the average distance between the outer boundary of the overflow region and the inner boundary of the receiving portion. Combinations of these ranges are also possible (e.g., greater than or equal to 1% and less than or equal to 70%, greater than or equal to 5% and less than or equal to 50%, or greater than or equal to 10% and less than or equal to 30%). Other ranges are also possible.

As described above, some overflow regions are at least partially unbounded in the porous, absorbent layers in which they are positioned. In such embodiments, overflow region may not have an outer boundary.

In some embodiments, a distance from an outer boundary of the sample collection region and an inner boundary of the receiving portion varies by a relatively small amount. For example, referring again to FIG. 13B, distance 2613 may vary by a relatively small amount.

In some embodiments, a distance from an outer boundary of the sample collection region to an inner boundary of the receiving portion varies by greater than or equal to 1%, greater than or equal to 2%, greater than or equal to 5%, greater than or equal to 8%, greater than or equal to 10%, greater than or equal to 12%, greater than or equal to 15%, greater than or equal to 18%, greater than or equal to 20%, greater than or equal to 22%, greater than or equal to 25%, greater than or equal to 30%, greater than or equal to 35%, greater than or equal to 40%, greater than or equal to 45%, greater than or equal to 50%, or greater than or equal to 60% of the average distance from an outer boundary of the sample collection region and an inner boundary of the receiving portion. In some embodiments, a distance from an outer boundary of the sample collection region to an inner boundary of the receiving portion varies by less than or equal to 70%, less than or equal to 60%, less than or equal to 50%, less than or equal to 45%, less than or equal to 40%, less than or equal to 35%, less than or equal to 30%, less than or equal to 25%, less than or equal to 22%, less than or equal to 20%, less than or equal to 18%, less than or equal to 15%, less than or equal to 12%, less than or equal to 10%, less than or equal to 8%, less than or equal to 5%, or less than or equal to 2% of the average distance from an outer boundary of the sample collection region and an inner boundary of the receiving portion. Combinations of these ranges are also possible (e.g., greater than or equal to 1% and less than or equal to 70%, greater than or equal to 5% and less than or equal to 50%, or greater than or equal to 10% and less than or equal to 30%). Other ranges are also possible.

The overlap portion may occupy any of a variety of appropriate portions of the area of the sample collection region. In some embodiments, an overlap portion occupies greater than or equal to 1%, greater than or equal to 2%, greater than or equal to 5%, greater than or equal to 10%, greater than or equal to 20%, greater than or equal to 30%, greater than or equal to 40%, greater than or equal to 50%, greater than or equal to 60%, greater than or equal to 70%, greater than or equal to 80%, greater than or equal to 90%, or greater than or equal to 95% of the area of the sample collection region. In some embodiments, an overlap portion occupies less than 100%, less than or equal to 95%, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, or less than or equal to 2% of the area of the sample collection region. Combinations of these ranges are also possible (e.g., greater than or equal to 1% and less than 100%, greater than or equal to 2% and less than or equal to 80%, or greater than or equal to 5% and less than or equal to 30%). Other ranges are also possible. In some embodiments, it may be advantageous for the overlap portion to occupy less than 100%, less than 90%, or less than 80% of the area of the sample collection region in order to permit at least some fluid (e.g., plasma) to travel through the sample collection region prior to its transmission to the overlap portion.

The receiving portion may occupy any of a variety of appropriate portions of the area of the overflow region. In some embodiments, a receiving portion occupies greater than or equal to 1%, greater than or equal to 2%, greater than or equal to 5%, greater than or equal to 10%, greater than or equal to 20%, greater than or equal to 30%, greater than or equal to 40%, greater than or equal to 50%, greater than or equal to 60%, greater than or equal to 70%, greater than or equal to 80%, greater than or equal to 90%, or greater than or equal to 95% of the area of the overflow region. In some embodiments, a receiving portion occupies less than 100%, less than or equal to 95%, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, or less than or equal to 2% of the area of the overflow region. Combinations of these ranges are also possible (e.g., greater than or equal to 1% and less than 100%, greater than or equal to 1% and less than or equal to 40%, greater than or equal to 5% and less than or equal to 30%, or greater than or equal to 10% and less than or equal to 20%). Other ranges are also possible.

The overflow portion may have any of a variety of appropriate shapes. For example, in some embodiments, the overflow portion has the shape of an annulus or an annular section, as shown in FIGS. 13A-13B. However, the disclosure is not so limited and any of a variety of appropriate shapes may be used.

It is also possible for a sample overflow region to be included in the first, absorbent layer. In such embodiments, the boundary enclosing the sample collection region may be interrupted by interstices through which fluid can flow, permitting it to be transmitted from the sample collection region to the sample overflow region. In some such embodiments, the sample collection region may be capable of being and/or configured to be torn away from the sample overflow region, thereby separating the contents of the sample collection region from the excess that has been transmitted into the sample overflow region. This may be facilitated by the presence of perforations in the interstices and/or along the boundary. When present in the interstices, such perforations may be incomplete (e.g., form an incomplete boundary) and/or allow for partial fluid flow therethrough.

According to some such embodiments, the sample overflow region does not include a receiving portion and the sample collection region does not include an overlap portion, because the sample overflow region is part of the same absorbent layer as the sample collection region. FIG. 13D presents a schematic, top-view illustration of an absorbent layer 305 comprising a sample collection region 307 that is bordered by a sample overflow region 368 that is also part of absorbent layer 305. Sample collection region 307 is bounded by boundary 321, which is perforated by perforations 344. Fluid may be transmitted from sample collection region 307 to sample overflow region 368 between perforations 344.

A sample overflow region in the same layer as the sample collection region may border the entire boundary of the sample collection region, or may border a fraction of the boundary of the sample collection region. Portions of the boundary of the sample collection region that are not bordered by the sample overflow region may instead be bordered, for example, by a barrier, by a gap, or by a layer edge, across which no fluid may be transmitted.

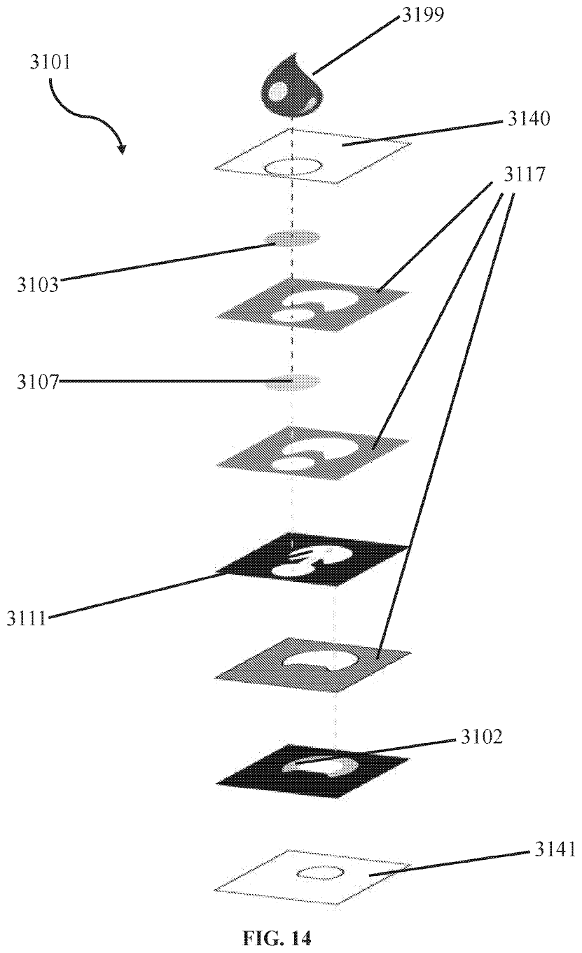

A sample overflow region may border any appropriation proportion of the boundary of the sample collection region. In some embodiments, a sample overflow region borders greater than or equal to 1%, greater than or equal to 5%, greater than or equal to 10%, greater than or equal to 20%, greater than or equal to 30%, greater than or equal to 40%, greater than or equal to 50%, greater than or equal to 60%, greater than or equal to 70%, greater than or equal to 80%, greater than or equal to 90%, or greater than or equal to 95% of the boundary of the sample collection region. In some embodiments, a sample overflow region borders less than or equal to 100%, less than or equal to 95%, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, or less than or equal to 5%, of the boundary of the sample collection region. Combinations of these ranges are also possible (e.g., greater than or equal to 1% and less than or equal to 100%, greater than or equal to 20% and less than or equal to 100%, or greater than or equal to 20% and less than or equal to 80%). Other ranges are also possible. FIG. 14 presents an exploded-perspective schematic illustration of a non-limiting article 3101 comprising an overflow region 3102, according to some embodiments. As shown, overflow region 3102 overlaps sample collection region 3111 of a second absorbent layer which is disposed beneath filters 3103 and 3107 configured to receive blood sample 3199. Also shown are portions 3040 and 3041 of the support structure, and adhesive layers 3117, which are configured to maintain the relative positions of article layers.

In some embodiments, the article comprises one or more filters. A filter may be configured to separate components of the fluid sample from each other. Some of the components of the fluid sample may be retained by the filter (e.g., on one side of the filter) while other components pass through the filter. For instance, an article may comprise a filter configured to separate blood cells from plasma. Plasma in a blood sample may flow through the filter (e.g., and into one or more channels of the article) while blood cells are retained by the filter. The plasma passing through the filter may flow to one or more sample collection regions, resulting in the formation of samples at the sample collection regions that comprise plasma and either lacking blood cells or include a relatively small amount of blood cells. Samples rich in plasma and poor in blood cells (or lacking blood cells) may be advantageous for blood tests sensitive to plasma components.

A filter may be configured to retain blood cells. For example, in some embodiments, the article comprises a first filter configured to separate blood cells from plasma. In some embodiments, the article comprises a second filter also configured to separate blood cells from plasma. The first and second filters may be configured to separate the same types of blood cells from plasma or may be configured to separate different types of blood cells from plasma. For instance, in some embodiments, an article comprises a first filter that is configured to separate white blood cells and/or leukocytes from plasma and a second filter configured to separate red blood cells and/or platelets from plasma. According to some embodiments, the second filter is disposed beneath the first filter. The first filter and the second filter may be fluidically connected. For example, the disposition of the second filter beneath the first filter may, in some cases, fluidically connect the first filter and the second filter. In some embodiments, the filter (e.g., the first filter and/or the second filter) is fluidically connected to an absorbent layer. For example, the second filter may be disposed on top of a portion of an absorbent layer (e.g., a filter reception region, as described below). In some embodiments, the first filter may be fluidically connected to the sample collection region via the second filter.

In some embodiments, a method comprises passing a blood sample through the first filter. Passing a blood sample through the first filter may produce a blood sample with reduced blood cells. In some embodiments, the method further comprises passing the blood sample with reduced blood cells to through a second filter. Passing the blood sample with reduced blood cells from the first filter through the second filter may produce a blood sample with further reduced blood cells, in some embodiments. The method may further comprise passing a blood sample (e.g., a blood sample with reduced blood cells, or a blood sample with further reduced blood cells) from a filter into an absorbent layer. For example, in some embodiments, the method comprises passing the blood sample with further reduced red blood cells into the absorbent layer. Alternately, in some embodiments, the method comprises passing the blood sample with reduced blood cells from the first filter directly into the absorbent layer. For example, in some embodiments, the method comprises passing a blood sample with further reduced red blood cells into absorbent layer 105 in FIG. 1.

The filters in the article may be in any suitable order. In some embodiments, the second filter is positioned between the first filter and the absorbent layer. For example, in FIG. 2, in accordance with some embodiments, second filter 207 is positioned between first filter 203 and absorbent layer 205. In some embodiments, the first filter is positioned between the second filter and absorbent layer.

In some embodiments, there are no intervening layers between the first filter and second filter and/or between the second filter and absorbent layer. For example, in FIG. 2, in accordance with some embodiments, there are no intervening layers between first filter 203 and second filter 207 or between second filter 207 and filter reception region 209 of absorbent layer 205. Without wishing to be bound by theory, it is believed that direct contact between the layers (e.g., the second and absorbent layer) improves the transport speed by increasing capillary action. However, in some embodiments, a small gap (e.g., to accommodate an adhesive) may be used.

In some embodiments, the filters are adjacent to one another. In some embodiments, a filter is adjacent to an absorbent layer. As used herein, when a layer is referred to as being “adjacent” another layer, it can be directly adjacent on the layer, or an intervening layer also may be present. A layer that is “directly adjacent” another layer is positioned with respect to the layer such that no intervening layer is present.

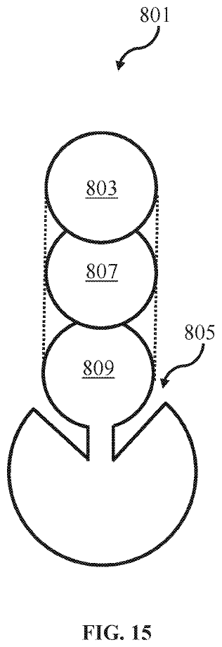

In some embodiments, some or all of the filters may be stacked coaxially, such that a vertical stack is formed. FIG. 15 presents an exploded perspective schematic illustration of an exemplary article 801 in which a vertical stack (indicated by dashed lines) is formed by the filters. For example, in FIG. 15, first filter 803 and second filter 807 are stacked coaxially, such that a vertical stack is formed. In some embodiments, some or all of the filters of the article (e.g., the first filter, the second filter) are coaxial with a filter reception region of an absorbent layer of the article. For example, referring again to FIG. 15, first filter 803 and second filter 807 are coaxial with filter reception region 809 of absorbent layer 805. Without wishing to be bound by theory, it is believed that the vertical stacking reduces the time required for separation.

As shown in FIG. 15, the filters and filter reception region may have any of a variety of appropriate forms. For example, in FIG. 15, first filter 803, second filter 807, and filter reception region 809 have a circular form. The disclosure is not thus limited. In some embodiments, the filters described herein are discrete layers.

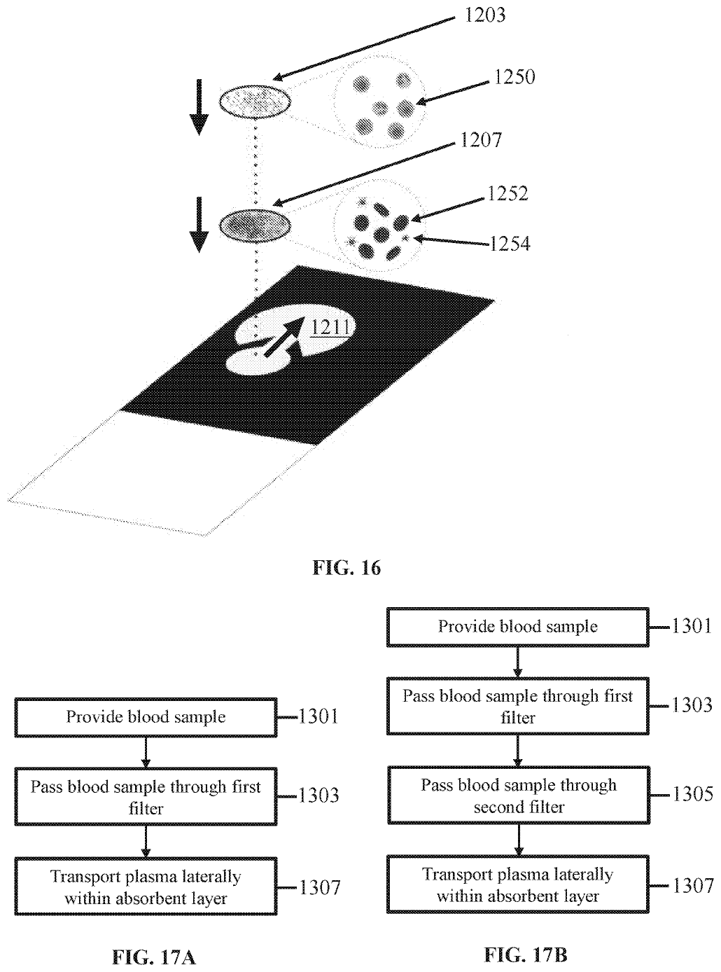

In some embodiments, the method of passing fluid through the filters (e.g., passing the blood sample across the first filter, passing the blood sample with reduced blood cells across the second filter, and/or passing the blood sample with further reduced blood cells into the absorbent layer) is passive. For example, in some embodiments, the method is done solely with the use of gravity and/or capillary action. For example, FIG. 16 illustrates separation of a blood sample by an exemplary article, according to some embodiments, where the sample is separated purely by gravity and capillary action. As indicated, the blood sample deposited on the first filter is drawn vertically through first filter 1203 and second filter 1207 (as indicated by the black arrows) and subsequently laterally transported into sample collection region 1211. An exemplary method representing this process is illustrated in the flow-chart of FIG. 17A. As shown in FIG. 17A, in some embodiments, in step 1301, a blood sample is provided to the article. Next, according to some embodiments, in step 1303, the blood sample is passed through a first filter of the article. This may produce a blood sample having reduced blood cells. Depending on the embodiment, the blood sample may be passed through a second filter to produce a sample with further reduced blood cells (e.g., a plasma). For example, FIG. 17B presents the exemplary method, wherein the blood is first passed through the first filter (step 1303) and the second passed through a filter (step 1305). This may, advantageously, produce purer plasma than could be achieved by passing the blood sample through a single filter. Finally, the plasma is separated laterally within the absorbent layer, in some embodiments, as shown in step 1307.

In some embodiments, the method (e.g., passing the blood sample across the first filter, passing the blood sample with reduced red blood cells across the second filter, and/or passing the blood sample with further reduced red blood cells into the absorbent layer) is rapid. In some embodiments, the method, starting with providing the blood sample to the article and concluding when the lateral transport of plasma within the absorbent layer ceases, is accomplished within less than or equal to 30 minutes, less than or equal to 20 minutes, less than or equal to 15 minutes, less than or equal to 10 minutes, less than or equal to 5 minutes, less than or equal to 3 minutes, or less than or equal to 2 minutes. In some embodiments, the method is accomplished within greater than or equal to 30 seconds, greater than or equal to 1 minute, greater than or equal to 2 minutes, greater than or equal to 3 minutes, or greater than or equal to 5 minutes. Combinations of these ranges are also possible (e.g., greater than or equal to 30 second and less than or equal to 10 minutes, or greater than or equal to 30 seconds and less than or equal to 5 minutes). Other ranges are also possible.

In some embodiments, the method (e.g., passing the blood sample across the first filter, passing the blood sample with reduced red blood cells across the second filter, and/or passing the blood sample with further reduced red blood cells into the absorbent layer) has a high separation efficiency. In some embodiments, the separation efficiency is greater than or equal to 10%, greater than or equal to 15%, greater than or equal to 20%, greater than or equal to 25%, greater than or equal to 30%, greater than or equal to 35%, greater than or equal to 40%, greater than or equal to 45%, greater than or equal to 50%, or greater than or equal to 55%. In some embodiments, the separation efficiency is less than or equal to 100%, less than or equal to 90%, less than or equal to 80%, less than or equal to 70%, less than or equal to 60%, less than or equal to 55%, less than or equal to 50%, less than or equal to 45%, less than or equal to 40%, less than or equal to 35%, or less than or equal to 30%. Combinations of these ranges are also possible (e.g., greater than or equal to 10% and less than or equal to 100%, greater than or equal to 10% and less than or equal to 60%, or greater than or equal to 30% and less than or equal to 55%). Other ranges are also possible.

As used herein, the separation efficiency is the percentage of collected purified plasma volume compared to the total theoretical plasma volume. The total theoretical plasma volume is based on the measured hematocrit value and input sample volume. For example, if a 100 microliter sample has a measured hematocrit value of 50%, then the total theoretical plasma volume is 50 microliters. If 40 microliters of purified plasma were collected, the separation efficiency would be 80%, since 40 microliters is 80% of 50 microliters.

As described above, in some embodiments, an article comprises one or more filters. General properties that may be applicable to some or all of the filters are provided below. Additional properties that may be particularly characteristic of one or more filters are described elsewhere herein with respect to such filter(s).

In some embodiments, an article comprises a filter that is configured to retain greater than or equal to 80%, greater than or equal to 85%, greater than or equal to 90%, greater than or equal to 95%, greater than or equal to 97%, greater than or equal to 99%, greater than or equal to 99.5%, or greater than or equal to 99.9% of the blood cells in blood that it filters. The filter may be configured to retain less than or equal to 100%, less than or equal to 99.9%, less than or equal to 99.5%, less than or equal to 99%, less than or equal to 97%, less than or equal to 95%, less than or equal to 90%, or less than or equal to 85% of the blood cells in blood that it filters. Combinations of the above-referenced ranges are also possible (e.g., greater than or equal to 80% and less than or equal to 100%, or greater than or equal to 90% and less than or equal to 100% of the blood cells that it filters). Other ranges are also possible.

Some methods may comprise passing blood through a filter, and it should be understood that these methods may comprise retaining a percentage of blood cells in one or more of the ranges described above on a first side of the filter (e.g., a side adjacent an environment external to the article). The percentage of blood cells retained by the filter may be determined by: (1) measuring the number of blood cells in a blood sample; (2) passing the blood sample through the filter; (3) measuring the number of blood cells in the blood sample after passage through the filter; (4) calculating a ratio of the number of blood cells in the blood sample after passage through the filter to the number of blood cells in the blood sample prior to passage through the filter; and (5) calculating the percentage of blood cells retained by the filter based on the ratio calculated in step (4).

In some embodiments, an article comprises a filter configured to filter certain types of blood cells from blood. The filter may be configured to pass some types of cells therethrough, and/or may be configured to also filter out other types of cells. For instance, some filters may be configured to retain white blood cells from blood while passing red blood cells and platelets therethrough (or vice versa). For example, referring again to FIG. 16, in some embodiments first filter 1203 is configured to retain white blood cells 1250 and second filter 1207 is configured to retain red blood cells 1252 and platelets 1254, as shown. It should be understood that the ranges described above may refer to the percentage of the total number of blood cells retained by the filter or may refer to the percentage of any specific type of blood cells retained by the filter (e.g., the percentage of white blood cells retained by the filter, the percentage of red blood cells retained by the filter).

In some embodiments, an article comprises a filter that is hydrophilic. The filter may have a water contact angle of less than or equal to 90°, less than or equal to 85°, less than or equal to 80°, less than or equal to 75°, less than or equal to 70°, less than or equal to 65°, less than or equal to 60°, less than or equal to 55°, less than or equal to 50°, less than or equal to 45°, less than or equal to 40°, less than or equal to 35°, less than or equal to 30°, less than or equal to 25°, less than or equal to 20°, less than or equal to 15°, less than or equal to 10°, or less than or equal to 5°. The filter may have a water contact angle of greater than or equal to 0°, greater than or equal to 5°, greater than or equal to 10°, greater than or equal to 15°, greater than or equal to 20°, greater than or equal to 25°, greater than or equal to 30°, greater than or equal to 35°, greater than or equal to 40°, greater than or equal to 45°, greater than or equal to 50°, greater than or equal to 55°, greater than or equal to 60°, greater than or equal to 65°, greater than or equal to 70°, greater than or equal to 75°, greater than or equal to 80°, or greater than or equal to 85°. Combinations of the above-referenced ranges are also possible (e.g., less than or equal to 900 and greater than or equal to 0°). Other ranges are also possible.

The water contact angle of a filter may be measured using ASTM D5946-04, which comprises positioning a water droplet on a plane solid surface of the filter. The water contact angle is the angle between the plane solid surface of the filter and the tangent line drawn to the water droplet surface at the three-phase point. A contact angle meter or goniometer can be used for this determination. In some embodiments, the hydrophilicity of the filter may be such that a water droplet placed on the surface completely wets the surface (e.g., the water droplet is completely absorbed into the material, making the water contact angle 0°). In some embodiments, an article may comprise a filter that is hydrophobic. The hydrophobic filter may have a water contact angle outside the ranges described above.

Filters may be porous, having porosities depending on the filter types and filter materials described below. Filters that are porous may comprise pores with a variety of suitable shapes. In some embodiments, a filter comprises asymmetric pores. The asymmetric pores may have a diameter that varies across the filter. The asymmetric pores may have a larger diameter on a first side of the filter (e.g., a side adjacent to an environment external to the article, a side configured to receive a fluid sample from an environment external to the article) and a smaller diameter on a second side of the filter (e.g., a side opposite the first side, a side adjacent to an absorbent layer, a side adjacent to a layer comprising one or more channels and/or one or more sample collection regions). A filter may comprise pores with a ratio of largest diameter (e.g., diameter of the portion of the pore adjacent to a first side of the filter) to smallest diameter (e.g., diameter of the portion of the pore adjacent to the opposite side of the filter) of greater than or equal to 1:1, greater than or equal to 1.1:1, greater than or equal to 1.2:1, greater than or equal to 1.5:1, greater than or equal to 2:1, greater than or equal to 2.2:1, greater than or equal to 2.5:1, greater than or equal to 3:1, or greater than or equal to 4:1. A filter may comprise pores with a ratio of largest diameter to smallest diameter of less than or equal to 5:1, less than or equal to 4:1, less than or equal to 3:1, less than or equal to 2.5:1, less than or equal to 2.2:1, less than or equal to 2:1, less than or equal to 1.5:1, less than or equal to 1.2:1, or less than or equal to 1.1:1. Combinations of the above-referenced ranges are also possible (e.g., greater than or equal to 1:1 and less than or equal to 5:1). Other ranges are also possible. The variation in pore diameter across a pore may be determined by electron microscopy.

In some embodiments, an article comprises a filter that is reversibly attached to another layer of the article. The filter may be capable of being removed from the first filter by hand (e.g., by peeling), without the use of specialized tools, and/or without destroying the first filter. For instance, the filter may be reversibly attached to the article by way of an adhesive that allows delamination of the filter from the article. Non-limiting examples of suitable adhesives include tapes, spray-on adhesives, double-sided films, screen-printed glues, and polymeric adhesives. In some embodiments, the filter may be permanently attached to the article (e.g., attached in a manner other than reversibly, such as integrally attached to the article). Permanent or integral attachment may be facilitated by the use of permanent adhesives.

In some embodiments, one or more layers is adhered to one or more layers in such a way that they can be pulled apart manually without damaging one or more the layers. As an example, in some embodiments, the first filter is adhered to the second filter such that they cannot be pulled apart manually without damaging one or more of the layers. As another example, in some embodiments, the second filter is adhered to the absorbent layer in such a way that they can be pulled apart manually without damaging one or more the layers. In some embodiments, the second filter is adhered to the absorbent layer in such a way that they can be pulled apart manually, without having to use so much force that it will disrupt the first filter, but such that the second filter and absorbent layer do not come apart during use (e.g., during blood separation). Separation of filters may advantageously facilitate analysis of cellular material from the filters, as described in greater detail below.

In some embodiments, the filters are positioned such that a sample comprising blood cells and/or cellular material can be recovered therefrom. For example, the filters may be configured to be removed (e.g., using tweezers, or using a punch).

The areas of the filters (e.g., a first filter and/or a second filter) in a plane perpendicular to the flow-through direction may generally be selected as desired. The area of the filter may be greater than or equal to 0.075 cm², greater than or equal to 0.1 cm², greater than or equal to 0.2 cm², greater than or equal to 0.5 cm², greater than or equal to 1 cm², greater than or equal to 1.5 cm², or greater. In some embodiments, the area of the filter is less than or equal to 10 cm², less than or equal to 5 cm², less than or equal to 2 cm², less than or equal to 1.5 cm², less than or equal to 1 cm², or less. Combinations of these ranges are possible (e.g., greater than or equal to 0.075 cm² and less than or equal to 10 cm², greater than or equal to 0.1 cm² and less than or equal to 5 cm 2, or greater than or equal to 0.2 cm² and less than or equal to 2 cm²). Other ranges are also possible.

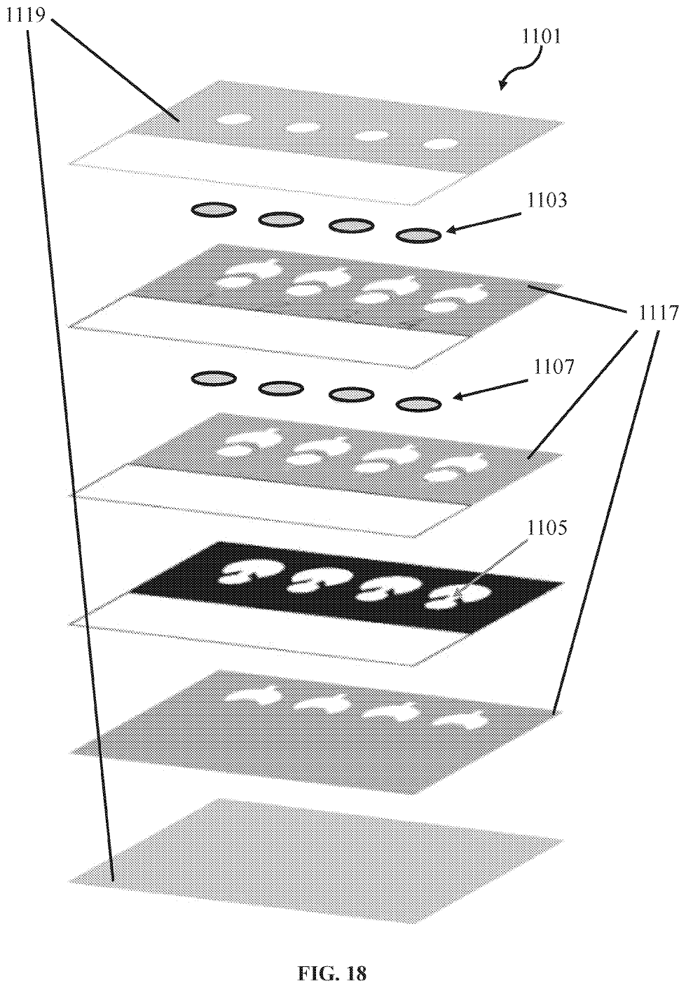

In some embodiments, the article comprises multiple, laterally offset filters. For example, laterally offset filters may be fluidically connected with sample collection regions that are laterally offset from each other. Advantageously, this may allow the article may be configured to separate more than one blood sample, e.g., by placing separate blood samples on separate portions of the article, to separate the blood samples into separate sample collection regions. FIG. 18 provides an exemplary, schematic, perspective illustration of article 1101 comprising multiple, laterally offset first filters 1103, laterally offset second filters 1107, and laterally offset absorbent layers 1105, according to some embodiments. In this example, the article is held together using adhesive layers 1117 and is supported by support structure 1119. Using article 1101, multiple blood samples can be separated by contacting each blood sample to different first filter 1103.

As described above, in some embodiments, an article described herein comprises a first filter. The first filter may be the only filter in the article, may be an uppermost filter in the article, and/or may be positioned in another suitable position. Further details regarding the first filter are provided below.

In some embodiments, the first filter comprises fiberglass, polyester, polyethersulfone, and/or nylon. In some embodiments, the polyester comprises a treated polyester, such as Leukosorb. The first filter may be fibrous or non-fibrous. For instance, it may comprise a fibrous membrane (e.g., comprising fibers including one or more of the above-referenced materials) and/or a mesh (e.g., comprising one or more of the above-referenced materials).

In some embodiments, the first filter is porous. In some embodiments, the mode pore size of the first filter is greater than or equal to 1 micron, greater than or equal to 2 microns, greater than or equal to 3 microns, greater than or equal to 4 microns, greater than or equal to 5 microns, greater than or equal to 8 microns, greater than or equal to 10 microns, or greater than or equal to 15 microns. In some embodiments, the mode pore size of the first filter is less than or equal to 30 microns, less than or equal to 25 microns, less than or equal to 20 microns, less than or equal to 15 microns, less than or equal to 10 microns, less than or equal to 9 microns, less than or equal to 8 microns, less than or equal to 7 microns, less than or equal to 6 microns, or less than or equal to 5 microns. Combinations of these ranges are also possible (e.g., greater than or equal to 1 micron and less than or equal to 30 microns, greater than or equal to 1 micron and less than or equal to 6 microns, greater than or equal to 2 microns and less than or equal to 25 microns, or greater than or equal to 8 microns and less than or equal to 20 microns). Other ranges are also possible.

One of ordinary skill could determine the mode pore size of a first filter using mercury intrusion porosimetery.

In some embodiments, the first filter can have a variety of suitable thicknesses. In some embodiments, the first filter has a small thickness so that the separation will be quicker. In some embodiments, the thickness of the first filter is greater than or equal to 150 microns, greater than or equal to 200 microns, greater than or equal to 250 microns, greater than or equal to 300 microns, or greater than or equal to 350 microns. In some embodiments, the thickness of the first filter is less than or equal to 800 microns, less than or equal to 700 microns, less than or equal to 600 microns, less than or equal to 500 microns, less than or equal to 400 microns, or less than or equal to 300 microns. Combinations of these ranges are also possible (e.g., greater than or equal to 150 microns and less than or equal to 800 microns, less than or equal to 250 microns and less than or equal to 650 microns, or greater than or equal to 350 microns and less than or equal to 500 microns). Other ranges are also possible.

In some embodiments, the first filter has a high loading capacity, such that it is configured to receive a blood sample with a substantial volume. In some embodiments, the blood sample is greater than or equal to 25 microliters, greater than or equal to 30 microliters, greater than or equal to 40 microliters, greater than or equal to 50 microliters, greater than or equal to 60 microliters, greater than or equal to 70 microliters, greater than or equal to 80 microliters, greater than or equal to 90 microliters, greater than or equal to 100 microliters, greater than or equal to 125 microliters, greater than or equal to 150 microliters, greater than or equal to 200 microliters, or greater than or equal to 250 microliters. In some embodiments, the loading capacity of the first filter is less than or equal to 500 microliters, less than or equal to 400 microliters, less than or equal to 300 microliters, less than or equal to 250 microliters, less than or equal to 200 microliters, less than or equal to 150 microliters, less than or equal to 125 microliters, less than or equal 100 microliters, less than or equal 90 microliters, less than or equal 80 microliters, or less than or equal 70 microliters. Combinations of these ranges are also possible (e.g., 25-500 microliters, 40-250 microliters, or 50-200 microliters). Other ranges are also possible.

The loading capacity of a filter may be determined by identification of the maximum volume of blood that can be applied without evidence of substantial hemolysis. Hemolysis may be detected by quantifying an amount of hemoglobin present in the filtered blood using Drabkin's assay. Hemolysis may be substantial if a concentration of hemoglobin in a filtered sample is at least 1%, at least 5%, at least 10%, at least 50%, or at least 100% greater than would be expected from a sample free of hemolysis byproducts.

In some embodiments, passing the blood sample across the first filter produces a blood sample with reduced red blood cells. In some embodiments, the red blood cells are reduced by greater than or equal to 20%, greater than or equal to 30%, greater than or equal to 40%, greater than or equal to 50%, greater than or equal to 60%, greater than or equal to 70%, greater than or equal to 80%, or greater than or equal to 90% of those in the blood sample. In some embodiments, the red blood cells are reduced by less than or equal to 100%, less than or equal to 90%, less than or equal to 80%, less than or equal to 70%, less than or equal to 60%, less than or equal to 50%, less than or equal to 40%, or less than or equal to 30% of those in the blood sample. Combinations of these ranges are also possible (e.g., greater than or equal to 20% and less than or equal to 100%, greater than or equal to 30% and less than or equal to 80%, or greater than or equal to 40% and less than or equal to 60%). Other ranges are also possible.

In some embodiments, the first filter reduces the level of red blood cells in the blood sample by size exclusion and/or electrostatic interactions.

In some embodiments, the first filter reduces the level of white blood cells. In some embodiments, the white blood cells are reduced by greater than or equal to 20%, greater than or equal to 30%, greater than or equal to 40%, greater than or equal to 50%, greater than or equal to 60%, greater than or equal to 70%, greater than or equal to 80%, or greater than or equal to 90% of those in the blood sample. In some embodiments, the white blood cells are reduced by less than or equal to 100%, less than or equal to 90%, less than or equal to 80%, less than or equal to 70%, less than or equal to 60%, less than or equal to 50%, less than or equal to 40%, or less than or equal to 30% of those in the blood sample. Combinations of these ranges are also possible (e.g., greater than or equal to 20% and less than or equal to 100%, greater than or equal to 40% and less than or equal to 90%, or greater than or equal to 60% and less than or equal to 80%). Other ranges are also possible.

In some embodiments, the first filter reduces the level of white blood cells in the blood sample by size exclusion and/or electrostatic interactions.

Without wishing to be bound by theory, it is believed that use of the first filter facilitates quick removal of a significant portion of the red blood cells, such that the second filter is less likely to get clogged and/or is less likely to cause hemolysis. In some embodiments, an article comprising a first filter article can have a relatively higher loading capacity without requiring lengthy times for separation. The reduced risk of clogging may be associated with the filtering of some of the blood cells by the first filter (reducing the number of blood cells reaching the second filter and/or the absorbent layer) and the relatively larger pore size of the first filter.

One of ordinary skill could measure the reduction in the level of white blood cells produced by a filter (e.g., a first, filter, a second filter) using flow cytometry. Similarly, one of ordinary skill could measure the reduction in the level of red blood cells produced by a filter (e.g., a first, filter, a second filter) using flow cytometry.

As described above, in some embodiments, an article described herein comprises a second filter. The second filter may be the only filter in the article, may be positioned beneath a first filter, may be positioned above an absorbent layer, and/or may be positioned in another suitable position. Further details regarding the second filter are provided below.

In some embodiments, the second filter comprises a polymer. The polymer may comprise an asymmetric polysulfone. For example, in some embodiments, the second filter comprises polyether sulfone. The second filter may be fibrous or non-fibrous. As an example of the latter, in some embodiments, the second filter comprises a plasma separation membrane. Non-limiting examples of suitable plasma separation membranes include Pall plasma separation membranes (e.g., a Pall Vivid plasma separation membrane (e.g., grade GX and/or grade GF)), Kinbio plasma separation membranes, and/or Cobetter plasma separation membranes.

In some embodiments, the second filter is porous. In some embodiments, the mode pore size of the second filter is greater than the mode pore size of the first filter. In some embodiments, the mode pore size of the second filter is smaller than the mode pore size of the first filter. In some embodiments, articles wherein the second filter comprises a pore size smaller than the first filter may, advantageously, retain larger cells (e.g., leukocytes such as white blood cells) in the first filter, while retaining smaller cells (e.g., red blood cells and/or platelets) in the second filter. Retaining different cells in different filters may advantageously reduce pore clogging in both filters, reducing shear-forces of fluid passing through pores, and thereby reducing cell lysis.

In some embodiments, the mode pore size of the second filter is greater than or equal to 0.1 microns, greater than or equal to 0.5 microns, greater than or equal to 1 micron, greater than or equal to 2 microns, greater than or equal to 3 microns, greater than or equal to 4 microns, greater than or equal to 5 microns, greater than or equal to 10 microns, or greater than or equal to 15 microns. In some embodiments, the mode pore size of the first filter is less than or equal to 100 microns, less than or equal to 75 microns, less than or equal to 50 microns, less than or equal to 30 microns, less than or equal to 25 microns, less than or equal to 20 microns, less than or equal to 15 microns, less than or equal to 10 microns, less than or equal to 9 microns, less than or equal to 8 microns, less than or equal to 7 microns, less than or equal to 6 microns, or less than or equal to 5 microns. Combinations of these ranges are also possible (e.g., greater than or equal to 0.1 microns and less than or equal to 100 microns, greater than or equal to 0.5 microns and less than or equal to 75 microns, or greater than or equal to 1 micron and less than or equal to 50 microns). Other ranges are also possible.

One of ordinary skill could determine the mode pore size of a second filter using scanning electron microscopy.

In some embodiments, a certain percentage of the pores of the second filter are below a certain size. In other words, the second filter includes a relatively low amount of pores that are relatively large. In some embodiments, the certain percentage (i.e., the percentage of pores that are below a certain size) is greater than or equal to 20%, greater than or equal to 30%, greater than or equal to 40%, greater than or equal to 50%, greater than or equal to 60%, greater than or equal to 70%, greater than or equal to 80%, or greater than or equal to 90%. In some embodiments, the certain percentage is less than or equal to 100%, less than or equal to 90%, less than or equal to 80%, less than or equal to 70%, less than or equal to 60%, less than or equal to 50%, less than or equal to 40%, or less than or equal to 30%. Combinations of these ranges are also possible (e.g., greater than or equal to 20% and less than or equal to 100%, greater than or equal to 50% and less than or equal to 100%, or greater than or equal to 90% and less than or equal to 100%). Other ranges are also possible.

In some embodiments, the certain size (i.e., the size that a certain percentage of the pores are below) is greater than or equal to 2 microns, greater than or equal to 3 microns, greater than or equal to 4 microns, greater than or equal to 5 microns, greater than or equal to 10 microns, or greater than or equal to 15 microns. In some embodiments, the certain size is less than or equal to 30 microns, less than or equal to 25 microns, less than or equal to 20 microns, less than or equal to 15 microns, less than or equal to 10 microns, less than or equal to 9 microns, less than or equal to 8 microns, less than or equal to 7 microns, less than or equal to 6 microns, or less than or equal to 5 microns. Combinations of these ranges are also possible (e.g., greater than or equal to 2 microns and less than or equal to 30 microns, or greater than or equal to 10 microns and less than or equal to 20 microns). Other ranges are also possible.

As further examples, in some embodiments, greater than or equal to 20% (e.g., greater than or equal to 50% or greater than or equal to 90%) of the pores of the second filter have a pore size of less than or equal to 20 microns (e.g., 10-20 microns).

In some embodiments, the second filter comprises a first surface and a second surface. In some embodiments, the first surface faces the first filter. In some embodiments, the second surface faces the absorbent layer. For example, in some embodiments, the second filter comprises first surface that faces the first filter 110, and a second surface that faces absorbent layer.

In some embodiments, second filter has a gradient in mode pore size between the first surface and the second surface. In some embodiments, a cross-section of the second filter between the first surface and the second surface has a mode pore size that is in between the mode pore size of the first surface and the mode pore size of the second surface.

In some embodiments, the second filter can have any of a variety of suitable thicknesses. In some embodiments, the thickness of the second filter is greater than or equal to 100 microns, greater than or equal to 150 microns, greater than or equal to 200 microns, or greater than or equal to 250 microns. In some embodiments, the thickness of the second filter is less than or equal to 500 microns, less than or equal to 400 microns, less than or equal to 350 microns, less than or equal to 300 microns, less than or equal to 250 microns, less than or equal to 200 microns, or less than or equal to 150 microns. Combinations of these ranges are also possible (e.g., greater than or equal to 100 microns and less than or equal to 500 microns, greater than or equal to 200 microns and less than or equal to 400 microns, or greater than or equal to 250 microns and less than or equal to 350 microns). Other ranges are also possible.

Some embodiments relate to a method that comprises passing the blood sample with reduced red blood cells across a second filter.

In some embodiments, passing the blood sample with reduced red blood cells across the second filter produces a blood sample with further reduced red blood cells. In some embodiments, the red blood cells are reduced by greater than or equal to 20%, greater than or equal to 30%, greater than or equal to 40%, greater than or equal to 50%, greater than or equal to 60%, greater than or equal to 70%, greater than or equal to 80%, greater than or equal to 90%, greater than or equal to 95%, or greater than or equal to 99% of those in the blood sample with reduced red blood cells. In some embodiments, the red blood cells are reduced by less than or equal to 100%, less than or equal to 99%, less than or equal to 95%, less than or equal to 90%, less than or equal to 80%, less than or equal to 70%, less than or equal to 60%, less than or equal to 50%, less than or equal to 40%, or less than or equal to 30% of those in the blood sample with reduced red blood cells. Combinations of these ranges are also possible (e.g., greater than or equal to 20% and less than or equal to 100%, greater than or equal to 40% and less than or equal to 100%, greater than or equal to 90% and less than or equal to 100%, or greater than or equal to 99% and less than or equal to 100%). Other ranges are also possible.

In some embodiments, the second filter further reduces the level of red blood cells in the blood sample with reduced red blood cells by size exclusion and/or electrostatic interactions.

In some embodiments, the second filter reduces the level of white blood cells. In some embodiments, the white blood cells are reduced by greater than or equal to 20%, greater than or equal to 30%, greater than or equal to 40%, greater than or equal to 50%, greater than or equal to 60%, greater than or equal to 70%, greater than or equal to 80%, or greater than or equal to 90% of those in the blood sample with reduced red blood cells. In some embodiments, the white blood cells are reduced by less than or equal to 100%, less than or equal to 90%, less than or equal to 80%, less than or equal to 70%, less than or equal to 60%, less than or equal to 50%, less than or equal to 40%, or less than or equal to 30% of those in the blood sample with reduced red blood cells. Combinations of these ranges are also possible (e.g., greater than or equal to 20% and less than or equal to 100%, greater than or equal to 40% and less than or equal to 100%, greater than or equal to 90% and less than or equal to 100%, or greater than or equal to 99% and less than or equal to 100%). Other ranges are also possible.

In some embodiments, the second filter reduces the level of white blood cells in the blood sample with reduced red blood cells by size exclusion and/or electrostatic interactions.

Without wishing to be bound by theory, it is believed that use of a second filter with a gradient in pore size reduces the risk of the second filter clogging and/or reduces the risk that the second filter will result in hemolysis.

In some embodiments, the second filter has a high loading capacity, such that it is configured to receive a blood sample with a substantial volume. In some embodiments, the loading capacity of the second filter is greater than or equal to 25 microliters, greater than or equal to 30 microliters, greater than or equal to 40 microliters, greater than or equal to 50 microliters, greater than or equal to 60 microliters, greater than or equal to 70 microliters, greater than or equal to 80 microliters, greater than or equal to 90 microliters, greater than or equal to 100 microliters, greater than or equal to 125 microliters, greater than or equal to 150 microliters, greater than or equal to 200 microliters, or greater than or equal to 250 microliters. In some embodiments, the loading capacity of the second filter is less than or equal to 500 microliters, less than or equal to 400 microliters, less than or equal to 300 microliters, less than or equal to 250 microliters, less than or equal to 200 microliters, less than or equal to 150 microliters, less than or equal to 125 microliters, less than or equal to 100 microliters, less than or equal to 90 microliters, less than or equal to 80 microliters, or less than or equal to 70 microliters. Combinations of these ranges are also possible (e.g., greater than or equal to 25 microliters and less than or equal to 500 microliters, greater than or equal to 40 microliters and less than or equal to 250 microliters, or greater than or equal to 50 microliters and less than or equal to 200 microliters). Other ranges are also possible.

In some embodiments, an article comprises an absorbent layer, as described above. The absorbent layer may be configured to transport fluid at a particular transport speed. For example, the absorbent layer may be configured to transport a blood sample at a transport speed of greater than or equal to 0.05 microliters/second, greater than or equal to 0.08 microliters/second, greater than or equal to 0.1 microliters/second, greater than or equal to 0.12 microliters/second, greater than or equal to 0.15 microliters/second, or greater. In some embodiments, the transport speed is less than or equal to 0.2 microliters/second, less than or equal to 0.15 microliters/second, less than or equal to 0.12 microliters/second, less than or equal to 0.1 microliters/second, less than or equal to 0.08 microliters/second, or less. Combinations of these ranges are possible (e.g., greater than or equal to 0.05 microliters/second and less than or equal to 0.2 microliters/second). Other ranges are also possible.

The absorbent layer may be hydrophilic (e.g., an absorbent layer may comprise a hydrophilic porous, absorbent material). All of the absorbent layer may be hydrophilic, or the absorbent layer may comprise a portion that is hydrophilic and a portion that is hydrophobic. For example, the absorbent layer may comprise a hydrophilic material (e.g., cellulose), that is templated with a hydrophobic material (e.g., wax).

An absorbent layer and/or a hydrophilic portion thereof may have a water contact angle of less than or equal to 90°, less than or equal to 85°, less than or equal to 80°, less than or equal to 75°, less than or equal to 70°, less than or equal to 65°, less than or equal to 60°, less than or equal to 55°, less than or equal to 50°, less than or equal to 45°, less than or equal to 40°, less than or equal to 35°, less than or equal to 30°, less than or equal to 25°, less than or equal to 20°, less than or equal to 15°, less than or equal to 10°, or less than or equal to 5°. An absorbent layer and/or a hydrophilic portion thereof may have a water contact angle of greater than or equal to 0°, greater than or equal to 5°, greater than or equal to 10°, greater than or equal to 15°, greater than or equal to 20°, greater than or equal to 25°, greater than or equal to 30°, greater than or equal to 35°, greater than or equal to 40°, greater than or equal to 45°, greater than or equal to 50°, greater than or equal to 55°, greater than or equal to 60°, greater than or equal to 65°, greater than or equal to 70°, greater than or equal to 75°, greater than or equal to 80°, or greater than or equal to 85°. Combinations of the above-referenced ranges are also possible (e.g., less than or equal to 900 and greater than or equal to 0°). Other ranges are also possible.

A hydrophobic portion of the absorbent layer may have a water contact angle of less than or equal to 180°, less than or equal to 175°, less than or equal to 170°, less than or equal to 165°, less than or equal to 160°, less than or equal to 155°, less than or equal to 150°, less than or equal to 145°, less than or equal to 140°, less than or equal to 135°, less than or equal to 130°, less than or equal to 125°, less than or equal to 120°, less than or equal to 115°, less than or equal to 110°, less than or equal to 105°, less than or equal to 100°, or less than or equal to 95°. The hydrophobic portion of the absorbent layer may have a water contact angle of greater than or equal to 90°, greater than or equal to 95°, greater than or equal to 100°, greater than or equal to 105°, greater than or equal to 110°, greater than or equal to 115°, greater than or equal to 120°, greater than or equal to 125°, greater than or equal to 130°, greater than or equal to 135°, greater than or equal to 140°, greater than or equal to 145°, greater than or equal to 150°, greater than or equal to 155°, greater than or equal to 160°, greater than or equal to 165°, greater than or equal to 170°, or greater than or equal to 175°. Combinations of the above-referenced ranges are also possible (e.g., less than or equal to 1800 and greater than or equal to 90°). Other ranges are also possible.

The water contact angle of a layer or portion thereof may be measured using ASTM D5946-04, as described above.

In some embodiments, the absorbent layer and/or one or more hydrophilic portions thereof comprises a cellulose-based material. The cellulose-based material may comprise cellulose derived from wood (e.g., it may be a wood-based material), cellulose derived from cotton (e.g., it may be a cotton-based material), cellulose derived from bacteria, and/or nitrocellulose. Nonlimiting examples of suitable cellulose-based absorbent layers include layers marketed commercially as Ahlstrom 226, Whatman 903, Munktell TFN, and Cytiva CF12.

In some embodiments, the absorbent layer and/or one or more hydrophilic portions thereof comprises a synthetic material and/or a glass. Non-limiting examples of suitable synthetic materials include poly(ether sulfone), polyesters, and nylons.

In some embodiments, the absorbent layer and/or one or more hydrophilic portions thereof comprises rayon and/or polyester (e.g., Kapmat). In some embodiments, the absorbent layer comprises a blend of rayon and polyester, such as a blend of rayon and polypropylene (e.g., ShamWow). The absorbent layer may be fibrous or non-fibrous.

Absorbent layers described herein may have any of a variety of designs. In some embodiments, an article comprises an absorbent layer comprising a fibrous material (e.g., a fibrous material comprising fibers formed from a cellulose-based material). The fibrous material may be a non-woven material, or may be a woven material. The fibers may have any of a variety of suitable diameters and distributions of diameters, and, if woven, may be woven in a variety of suitable weaves. In some embodiments, the non-woven material is a paper, such as a cellulose-based paper. A wide variety of commercially available cellulose-based papers may be employed, such as those manufactured by Whatman, those manufactured by Ahlstrom, and/or those manufactured by Munktell.

Fibrous materials may comprise fibers having any suitable average fiber diameter. The average fiber diameter of the fibers may be greater than or equal to 0.1 microns, greater than or equal to 0.2 microns, greater than or equal to 0.5 microns, greater than or equal to 1 micron, greater than or equal to 2 microns, greater than or equal to 5 microns, greater than or equal to 10 microns, greater than or equal to 15 microns, greater than or equal to 20 microns, greater than or equal to 25 microns, greater than or equal to 30 microns, greater than or equal to 40 microns, greater than or equal to 50 microns, greater than or equal to 60 microns, or greater than or equal to 70 microns. The average fiber diameter of the fibers may be less than or equal to 75 microns, less than or equal to 70 microns, less than or equal to 60 microns, less than or equal to 50 microns, less than or equal to 40 microns, less than or equal to 30 microns, less than or equal to 25 microns, less than or equal to 20 microns, less than or equal to 15 microns, less than or equal to 10 microns, less than or equal to 5 microns, less than or equal to 2 microns, less than or equal to 1 micron, less than or equal to 0.5 microns, or less than or equal to 0.2 microns. Combinations of the above-referenced ranges are also possible (e.g., greater than or equal to 0.1 micron and less than or equal to 75 microns). Other ranges are also possible. The average fiber diameter may be determined using electron microscopy.

Absorbent layers described herein may be porous. The absorbent layer may have any of a variety of suitable porosities. The porosity of the absorbent layer may be greater than or equal to 1 vol %, greater than or equal to 2 vol %, greater than or equal to 5 vol %, greater than or equal to 10 vol %, greater than or equal to 20 vol %, greater than or equal to 50 vol %, greater than or equal to 55 vol %, greater than or equal to 60 vol %, greater than or equal to 65 vol %, greater than or equal to 70 vol %, greater than or equal to 75 vol %, or greater than or equal to 80 vol %. The porosity of the absorbent layer may be less than or equal to 85 vol %, less than or equal to 80 vol %, less than or equal to 75 vol %, less than or equal to 70 vol %, less than or equal to 65 vol %, less than or equal to 60 vol %, less than or equal to 55 vol %, less than or equal to 50 vol %, less than or equal to 20 vol %, less than or equal to 10 vol %, less than or equal to 5 vol %, or less than or equal to 2 vol %. Combinations of the above-referenced ranges are also possible (e.g., greater than or equal to 1 vol % and less than or equal to 85 vol %, greater than or equal to 1 vol % and less than or equal to 80 vol %, or greater than or equal to 50 vol % and less than or equal to 80 vol %). Other ranges are also possible. The porosity of a material or a layer may be determined by mercury intrusion porosimetry.

As described above, in some embodiments, a portion of the pores in an absorbent layer may be filled with a hydrophobic material. In such instances, the porosities described above may independently characterize either the absorbent layer as a whole, one or more portions of the absorbent material for which the pores are unfilled, or all of the portions of the absorbent material whose pores remain unfilled.

In some embodiments, the absorbent layer is porous. In some embodiments, the absorbent layer has a mode pore size greater than or equal to 20 microns, greater than or equal to 25 microns, greater than or equal to 30 microns, greater than or equal to 35 microns, greater than or equal to 40 microns, greater than or equal to 50 microns, greater than or equal to 75 microns, greater than or equal to 90 microns, greater than or equal to 100 microns, or greater than or equal to 125 microns. In some embodiments, the absorbent layer has a mode pore size less than or equal to 150 microns, less than or equal to 125 microns, less than or equal to 100 microns, less than or equal to 90 microns, less than or equal to 75 microns, less than or equal to 50 microns, or less than or equal to 40 microns. Combinations of the above-referenced ranges are also possible (e.g., greater than or equal to 20 microns and less than or equal to 150 microns, greater than or equal to 75 microns and less than or equal to 125 microns, or greater than or equal to 90 microns and less than or equal to 100 microns). Other ranges are also possible. The mode pore size of an absorbent layer may be determined by mercury intrusion porosimetry.

As described above, absorbent layers comprise one or more regions (e.g., sample collection regions) and/or channels. For instance, an absorbent layer may comprise a filter reception region fluidically connected with an environment external to an article (e.g., a filter reception region configured to receive a fluid sample from the environment external to the article, such as after being passed through a filter from an environment external to the article), one or more channels, and/or one or more sample collection regions. In some embodiments, the filter reception region, the one or more channels, and/or the one or more sample collection regions may be positioned in absorbent layer.

Regions and/or channels may be formed in an absorbent layer by a variety of suitable methods. In some embodiments, the region (e.g., sample collection region, filter reception region) and/or the channel is bounded by a boundary, as described above. The boundary may be formed by a barrier. Alternatively, the boundary may be formed by a cut, gap, perforation, hole, or external boundary of the absorbent layer. In some embodiments, the boundary of a portion or channel may be of one or more types. For example, a boundary may comprise a portion that takes the form of a barrier, and a portion that takes the form of a gap. As a more specific example, at least a portion of the boundary of a region (e.g., a sample collection region) may be perforated, in some embodiments.

A barrier may be a barrier impermeable to fluids. For example, the barrier may be a spatial transition between a hydrophilic portion and a hydrophobic portion of the absorbent layer. In some embodiments, the barrier separates a hydrophobic material from a portion of the absorbent layer that is hydrophilic. According to some embodiments, at least a section of the sample collection region is surrounded by a hydrophobic material. By way of example, a barrier impermeable to a fluid may be infiltrated into portions of the layer and/or material to define channels and/or regions therein. This may be accomplished by, e.g., printing (e.g., wax printing, 3D-printing) and/or pattern transfer methods (e.g., by use of photoresists and/or UV-curable materials). The fluid to which the barrier is impermeable (e.g., a fluid sample, one or more components of a fluid sample) may, upon entering a channel and/or region defined by an impermeable barrier, be confined to portions of the layer and/or material to which it can flow through without crossing the impermeable barrier (e.g., channels and/or regions fluidically connected with the channel and/or region bounded by the impermeable barrier).

Barriers impermeable to a variety of fluids may be employed. In some embodiments, the fluid to which a barrier is impermeable is an aqueous fluid, such as a fluid of biological origin. Non-limiting examples of fluids of biological origin include blood (e.g., whole blood) and fluids derived from blood (e.g., plasma), cerebrospinal fluid, tissue biopsies, milk, wound exudate, saliva, tears, and urine. The barrier impermeable to a fluid may comprise a variety of suitable materials, non-limiting examples of which include waxes, polymers, and hydrophobic materials (e.g., hydrophobic waxes, hydrophobic polymers, other hydrophobic materials).

In some embodiments, an absorbent layer has an area in a lateral plane of the article. The area of the absorbent layer may be greater than or equal to 0.1 cm², greater than or equal to 0.2 cm², greater than or equal to 0.5 cm², greater than or equal to 1 cm², greater than or equal to 2 cm², or greater. In some embodiments, the area of the absorbent layer is less than or equal to 20 cm², less than or equal to 10 cm², less than or equal to 5 cm², less than or equal to 2 cm², less than or equal to 1.5 cm², less than or equal to 1 cm², or less. Combinations of these ranges are possible (e.g., greater than or equal to 0.075 cm² and less than or equal to 10 cm², greater than or equal to 0.1 cm² and less than or equal to 5 cm², or greater than or equal to 0.2 cm² and less than or equal to 2 cm²). Other ranges are also possible.

In some embodiments, the absorbent layer may have any of a variety of suitable absorbencies. In some embodiments, the absorbency is greater than or equal to 10 microliters/cm², greater than or equal to 14 microliters/cm², greater than or equal to 20 microliters/cm², greater than or equal to 30 microliters/cm², greater than or equal to 40 microliters/cm², greater than or equal to 50 microliters/cm², greater than or equal to 60 microliters/cm², greater than or equal to 70 microliters/cm², greater than or equal to 80 microliters/cm², greater than or equal to 90 microliters/cm², greater than or equal to 100 microliters/cm², greater than or equal to 125 microliters/cm², greater than or equal to 150 microliters/cm², greater than or equal to 175 microliters/cm², greater than or equal to 200 microliters/cm², greater than or equal to 250 microliters/cm², greater than or equal to 300 microliters/cm², or greater than or equal to 400 microliters/cm². In some embodiments, the absorbency is less than or equal to 600 microliters/cm², less than or equal to 550 microliters/cm², less than or equal to 500 microliters/cm², less than or equal to 450 microliters/cm², less than or equal to 400 microliters/cm², less than or equal to 300 microliters/cm², less than or equal to 250 microliters/cm², less than or equal to 200 microliters/cm², less than or equal to 175 microliters/cm², less than or equal to 150 microliters/cm², less than or equal to 100 microliters/cm², or less than or equal to 70 microliters/cm². Combinations of these ranges are also possible (e.g., greater than or equal to 10 microliters/cm² and less than or equal to 600 microliters/cm², greater than or equal to 10 microliters/cm² and less than or equal to 200 microliters/cm², or greater than or equal to 14 microliters/cm² and less than or equal to 70 microliters/cm²). Other ranges are also possible.

As used herein, the absorbency of an article and/or layer is determined by weighing the article and/or layer, saturating it in DI water for 30 seconds, weighing it again, determining the difference between the second weight and the first weight (i.e., the weight of the DI water absorbed), and then converting this weight to a volume of water (e.g., microliters) using the density of DI water at room temperature. The volume of DI water absorbed is then normalized by dividing by the surface area (e.g., cm²) of the article and/or layer.

In some embodiments, the absorbent layer is configured to absorb a variety of suitable fluids. Non-limiting examples of suitable fluids include water, blood plasma, saliva, tears, urine, wound exudate, and cerebrospinal fluid. In some embodiments, the absorbent layer is configured to absorb blood plasma.

In some embodiments, the absorbent layer may have any of a variety of suitable thicknesses. In some embodiments, the absorbent layer has a large thickness so that a large volume of fluid can be absorbed. In some embodiments, the thickness of the absorbent layer is greater than or equal to 100 microns, greater than or equal to 150 microns, or greater than or equal to 200 microns. In some embodiments, the thickness of the absorbent layer is less than or equal to 1000 microns, less than or equal to 900 microns, less than or equal to 700 microns, or less than or equal to 500 microns. Combinations of these ranges are also possible (e.g., greater than or equal to 100 microns and less than or equal to 1000 microns). Other ranges are also possible.

Some embodiments relate to a method that comprises removing the absorbent layer from a filter. For example, in some embodiments, such as some embodiments relating to articles initially having a structure as shown in FIG. 3, an absorbent layer may be removed from the second filter. In some embodiments, the absorbent layer is removed from the second filter by pulling it apart from the second filter. In some embodiments, the absorbent layer is pulled apart from the second filter manually (e.g., pulling it apart with tweezers). In some embodiments, the article comprises a boundary as described above. The boundary may, advantageously, improve handling of the sample collection region using tweezers. In some embodiments, pulling the tab may pull the absorbent layer apart from the second filter.

In some embodiments, the blood sample with further reduced red blood cells is stored inside the absorbent layer. In some embodiments, the blood sample with further reduced red blood cells is stored inside the absorbent layer in a wet state. In some embodiments, the blood sample with further reduced red blood cells is stored inside the absorbent layer in a dry state. For example, in some embodiments, the absorbent layer containing the blood sample with further reduced red blood cells is dried overnight. In some embodiments, the absorbent layer is dried overnight in a sealed container. In some embodiments, the sealed container comprises a desiccant.

In some embodiments, the dried absorbent layer is later rehydrated. In some embodiments, the dried absorbent layer is rehydrated by adding a solvent, such as an aqueous solution (e.g., an aqueous solution comprising a surfactant), a buffered solution (e.g., phosphate buffered saline), and/or water (e.g., DI water).

In some embodiments, two or more layers are adhered together. FIG. 18 shows one example of an article comprising several such layers. In FIG. 18, the article comprises adhesive layers 1117, which adhere first filter 1103 to second filter 1107, and which adhere second filter 1107 to absorbent layer 1105. In some embodiments, one or more layers are permanently adhered to one or more layers. In some embodiments, one or more layers are reversibly adhered to one or more layers. Examples of suitable methods of adhering layers include double-sided medical adhesive, liquid adhesive (e.g., adhesive spray), epoxies, film adhesives, pastes, sonic welding, and/or compression. In some embodiments, two or more layers are adhered together (and/or to a support structure) with an adhesive. Examples of suitable adhesives include double-sided medical adhesive, compression tape, 3M brand adhesives (e.g., 3M brand adhesive spray), and/or Flexcon brand adhesive. In some embodiments, the adhesive is placed on a surface of a layer. In some embodiments, the adhesive is placed around the perimeter of a layer to adhere it to another layer.

Some articles described herein comprise a support structure. In some embodiments, the support structure comprises a plastic, an acrylic, and/or a metal. In some embodiments, the support structure is a plastic scaffold or an acrylic scaffold. In some embodiments, the support structure is configured to maintain conformal contact between the absorbent layer and one or more layers (e.g., the second filter).

In some embodiments, the support structure is adjacent one or more layers. In some embodiments, the support structure is adjacent the first filter, second filter, and/or absorbent layer. In some embodiments, the support structure is in direct contact with one or more layers. In some embodiments, the support structure is in direct contact with the first filter, second filter, and/or absorbent layer. In some embodiments, the support structure is in direct contact with the second filter and absorbent layer. In some embodiments, the support structure is in direct contact with the absorbent layer.

In some embodiments, the support structure is adhered to one or more layers (e.g., the absorbent layer). Examples of suitable means to adhere (e.g., the support structure to one or more layers) are discussed elsewhere herein (e.g., in reference to adhering one layer to another layer). In some embodiments, the support structure is not adhered to one or more layers (e.g., not adhered to any layers). For example, in some embodiments, the article sits on the support structure.

In some embodiments, the support structure comprises a cavity. In some embodiments, the cavity is used for holding the article and/or one or more layers. In some embodiments, the cavity is circular, oval, square, rectangular, and/or diamond shaped. In some embodiments, the cavity is of a similar shape as a layer (e.g., a filter, an absorbent layer) of the article. For example, in some embodiments, the cavity and/or the cross-section are both circular, oval, square, rectangular, and/or diamond shaped.

In some embodiments, the depth of the cavity is less than the thickness of the support structure, such that, when viewed from above, a layer of the support structure is present throughout the surface area of the support structure. In some embodiments, the cavity is configured such that the article can sit inside the cavity. In some embodiments, the cavity is configured such that the article can sit inside the cavity, with the bottom surface of the absorbent layer in contact with the support structure.

In some embodiments, the cavity is present throughout the thickness of the support structure, such that, when viewed from above, the cavity is a hole in the support structure. In some embodiments, the cavity has different maximum horizontal dimensions at different thickness of the support structure. For example, in some embodiments, the cavity has a larger maximum horizontal dimension at one opening than at the other. In some embodiments, the larger maximum horizontal dimension at one opening is greater than or equal to the maximum horizontal dimension of the article and/or layer. In some embodiments, the smaller maximum horizontal dimension at the other opening is less than the maximum horizontal dimension of the article and/or layer. In some embodiments, the cavity is configured such that the article can sit inside the cavity. In some embodiments, the cavity is configured such that the article can sit inside the cavity, but the bottom surface of the absorbent layer is not in contact with the support structure. In some embodiments, the cavity is configured such that the article can sit inside the cavity, but the bottom surface of the absorbent layer is not in contact with the support structure, such that the absorbent layer can be removed from the article through the bottom of the support structure (e.g., through the opening with the smaller maximum horizontal dimension), while the remaining portions of the article can remain in the support structure.

In some embodiments, the cavity is configured such that the height of the edges (e.g., circumference) of the cavity prevent the article from significant horizontal movement, but the article can still be picked up vertically. In some embodiments, the height of the edges of the cavity are greater than or equal to ⅕ the thickness of a layer (e.g., the absorbent layer), greater than or equal to ¼ the thickness of a layer (e.g., the absorbent layer), greater than or equal to ⅓ the thickness of a layer (e.g., the absorbent layer), greater than or equal to ½ the thickness of a layer (e.g., the absorbent layer), or greater than or equal to the thickness of a layer (e.g., the absorbent layer). In some embodiments, the height of the edges of the cavity are less than or equal to 3 times the thickness of a layer (e.g., the absorbent layer), 2 times the thickness of a layer (e.g., the absorbent layer), the thickness of a layer (e.g., the absorbent layer), ½ the thickness of a layer (e.g., the absorbent layer), ⅓ the thickness of a layer (e.g., the absorbent layer), or ¼ the thickness of a layer (e.g., the absorbent layer). Combinations of these ranges are also possible (e.g., greater than or equal to ⅕ the thickness of a layer and less than or equal to 3 times the thickness of a layer). Other ranges are also possible.

In some embodiments, a method comprises collecting the blood sample with further reduced red blood cells from the absorbent layer. For example, the method may comprise extracting plasma from the sample collection region. In some embodiments, collecting the blood sample with further reduced red blood cells is done shortly after the blood sample with further reduced red blood cells is passed into the absorbent layer. In some embodiments, collecting the blood sample with further reduced red blood cells is done after the sample with further reduced blood cells has been stored inside the absorbent layer for a length of time. In some embodiments, the blood sample with further reduced red blood cells is collected from the absorbent layer greater than or equal to 1 minute, greater than or equal to 5 minutes, greater than or equal to 15 minutes, greater than or equal to 30 minutes, greater than or equal to 1 hour, greater than or equal to 5 hours, greater than or equal to 12 hours, greater than or equal to 1 day, greater than or equal to 3 days, greater than or equal to 1 week, greater than or equal to 1 month, greater than or equal to 6 months, or greater than or equal to 1 year after it has been passed into the absorbent layer. In some embodiments, the blood sample with further reduced red blood cells is collected from the absorbent layer less than or equal to 3 years, less than or equal to 2 years, less than or equal to 1 year, less than or equal to 6 months, less than or equal to 1 month, less than or equal to 1 week, less than or equal to 3 days, less than or equal to 1 day, less than or equal to 12 hours, less than or equal to 5 hours, less than or equal to 1 hour, less than or equal to 30 minutes, less than or equal to 15 minutes, or less than or equal to 5 minutes after it has been passed into the absorbent layer. Combinations of these ranges are also possible (e.g., greater than or equal to 1 minute and less than or equal to 3 years). Other ranges are also possible.

In some embodiments, the blood sample with further reduced red blood cells (e.g., the blood sample with further reduced red blood cells collected from the absorbent layer) can be collected in a short period of time. In some embodiments, the blood sample with further reduced blood cells can be collected in less than or equal to 30 minutes, less than or equal to 20 minutes, less than or equal to 15 minutes, less than or equal to 10 minutes, less than or equal to 5 minutes, less than or equal to 3 minutes, or less than or equal to 1 minute. In some embodiments, the blood sample with further reduced blood cells can be collected in greater than or equal to 30 seconds, greater than or equal to 1 minute, greater than or equal to 2 minutes, greater than or equal to 3 minutes, or greater than or equal to 5 minutes. Combinations of these ranges are also possible (e.g., 30 seconds to 30 minutes, or 30 seconds to 10 minutes). Other ranges are also possible.

In some embodiments, after collection, the blood sample with further reduced red blood cells (e.g., pure plasma) can be used in subsequent applications, such as in diagnostic health tests, clinical assay (e.g., clinical chemistry assays), immunoassays, rapid dipstick tests, cholesterol test, metabolite panels, serology for infectious diseases, therapeutic drug monitoring, ELISAs, ICP-AES, HPLC, and/or mass spectrometry. More non-limiting examples of subsequent applications for the blood sample with further reduced blood cells include polymerase chain reaction (PCR) applications (e.g., qPCR, RT-PCR, RT-qPCR) and isothermal amplification. As a non-limiting example, in some embodiments, the method may comprise determining an amount of a virus in the plasma. For example, the method may comprise determining an amount of an HIV virus (e.g., assaying HIV viral load). As another non-limiting example, the method may comprise detection of an analyte (e.g., within the plasma). Exemplary analytes include proteins, antibodies, hormones, metabolites, lipids, or drugs. The blood sample with further reduced red blood cells (e.g., pure plasma) may be analyzed using any appropriate technique, such as spectrophotometry, HPLC, spectrometry, electrophoresis, and/or chemiluminescence.

In some embodiments, the blood sample with further reduced red blood cells (e.g., the blood sample with further reduced red blood cells collected from the absorbent layer) has a volume that is a significant percentage of the volume of the initial blood sample (e.g., the blood sample prior to passage through the first filter), given that 20-60% of whole blood can be red blood cells. In some embodiments, the blood sample with further reduced red blood cells has a volume that is greater than or equal to 10%, greater than or equal to 12%, greater than or equal to 15%, greater than or equal to 17%, greater than or equal to 20%, greater than or equal to 25%, greater than or equal to 30%, greater than or equal to 35%, greater than or equal to 40%, greater than or equal to 45%, or greater than or equal to 50% of the volume of the initial blood sample. In some embodiments, the blood sample with further reduced red blood cells has a volume that is less than or equal to 80%, less than or equal to 70%, less than or equal to 60%, less than or equal to 50%, less than or equal to 40%, less than or equal to 30%, less than or equal to 25%, less than or equal to 20%, less than or equal to 17%, or less than or equal to 15% of the volume of the initial blood sample. Combinations of these ranges are also possible (e.g., greater than or equal to 10% and less than or equal to 80%, or greater than or equal to 10% and less than or equal to 40%). Other ranges are also possible.

In some embodiments, the blood sample with further reduced red blood cells (e.g., the blood sample with further reduced red blood cells collected from the absorbent layer or the blood sample) is pure (e.g., pure plasma and/or serum) and/or is free of red blood cells. In some embodiments, the blood sample with further reduced red blood cells has less than or equal to 5%, less than or equal to 4%, less than or equal to 3%, less than or equal to 2%, or less than or equal to 1% of the red blood cells in the initial blood sample (e.g., a whole blood sample).

In some, but not all, embodiments, the article and/or method has one or more advantages, such as short separation time, short collection time, ease of separation (e.g., without constant manual operation), ease of collection (e.g., without the use of high speed centrifuges), small surface area (e.g., small maximum horizontal dimension) of the article, ease of scaling up, ease of storage of the purified sample, large loading capacity, large volume recovery, low amounts of clogging of the article, low amounts of hemolysis in the recovered sample, high purity of the recovered sample, low amounts of mess (e.g., high containment of the blood within the article), low energy requirements, and/or ability to use whole blood samples without the need for dilution.

In some embodiments, cellular material retained by filters may be analyzed. For example, a method may comprise analyzing cellular material (e.g., genetic material) from the first and/or second filters. Without wishing to be bound by theory, collected cellular material produced during separation of the plasma from the blood cells may increase the local concentration of cellular material, advantageously improving detection. In some embodiments, analyzing cellular material from the first and/or second filters comprises amplifying genomes of cellular material present in the first and/or second filters.

In some embodiments, an article comprises one or more reagents. Each reagent and/or each combination of reagents may be suitable for any of a variety of purposes.

In some embodiments, an article comprises one or more reagents that improve analyte recovery. Such reagents and/or combinations of reagents may comprise blocking agents, stabilizing agents, denaturants, and/or wetting agents. Non-limiting examples of blocking agents, which may block non-specific binding sites, include albumin (e.g., bovine serum albumin), skim milk (e.g., in dehydrated form), and/or casein. Non-limiting examples of stabilizing agents, which may stabilize one or more analytes during article preparation and/or storage, include anti-coagulants (e.g., ethylenediaminetetraacetic acid (EDTA), heparin), salts (e.g., sodium chloride, ammonium sulfate, potassium chloride, sodium citrate), surfactants, sugars (e.g., sucrose, trehalose), albumin, and pH modifiers. Non-limiting examples of pH modifiers include sodium citrate and buffers (e.g., ammonium sulfate, acetate buffer, sodium citrate, phosphate buffered saline, a sodium carbonate buffer, tris buffer, and/or a HEPES buffer). Non-limiting examples of denaturants include sodium dodecylsulfate, urea, guanidinium thiocyanate, and lithium perchlorate. When present, a denaturant may, advantageously, denature an RNAse and/or a DNAse, thereby preserving the nucleic acid(s) the RNAse and/or DNAse would otherwise denature. Non-limiting examples of wetting agents include surfactants, such as O,O′-Bis(2-aminopropyl) propylene glycol-block-polyethylene glycol-block-polypropylene glycol (e.g., Jeffamine), poly(diallyldimethylammonium chloride), polyethylene glycol sorbitan monolaurate (e.g., Tween 20), sodium dodecylsulfate, polyoxyethylene (23) lauryl ether (e.g., Brij23, Brij 35, C12E23), and polyethylene glycol tert-octylphenyl ether (Triton X-100).

The article may include anti-clogging reagents. The anti-clogging reagents may prevent coagulation and/or agglutination of blood. The anti-clogging reagents may be anti-coagulants, salts, or pH modifiers, as described above. For example, non-limiting examples of anti-clogging agents include EDTA, heparin, sodium chloride, and potassium chloride.

In some embodiments, an article comprises one or more reagents that function as a preservative. Preservatives may encapsulate analytes upon drying, which may improve recovery upon rehydration. In some embodiments, such preservatives may rehydrate to form hydrogels that encapsulate one or more reagents. Non-limiting examples of preservatives include silk fibroin proteins and hydrogel precursors (e.g., pullulan, alginic acid).

In some embodiments, an article comprises one or more reagents that are oxidizing agent(s), such as ammonium persulfate. The oxidizing agent may act as a preservative by oxidizing lipoproteins. Non-limiting examples of oxidation agents for lipoprotein oxidation may include tert-butylhydroquinone, alpha-tocopherol; alpha-tocopheryl hydroquinone, alpha-tocopheryl quinone and derivates of those compounds.

In some embodiments, an article comprises one or more reagents that are reducing agent(s), such as ascorbic acid or vitamin E.

It is also possible for an article to comprise one or more reagents that are biologically active. For example, an article may comprise a cell lysis reagent (e.g., as saponin), a ligand configured to capture a species to be assayed (e.g., a monoclonal or a polyclonal antibody, a nanobody, an aptamer), an enzyme (e.g., RNAse, DNAse, horseradish peroxidase), and/or an enzyme inhibitor (e.g., a protease). Non-limiting examples of suitable antibodies include anti-pLDH (malaria), anti-p24 (HIV), anti-hCG (pregnancy), anti-CRP (acute phase injury), anti-NS1 (dengue) and anti-human IgG. A reagent may comprise an enzymatic substrate, such as acetylthiocholine chloride. When present, an aptamer may be conjugated to a species that may be easily detected, such as a colored particle (e.g., a colloidal gold nanoparticle), a colorimetric reagent (e.g., 3,3′,5,5′-Tetramethylbenzidine (TMB); potassium iodide (KI); 2,2′-azino-bis(3-ethylbenzothiazoline-6-sulfonic acid (ABTS); 3,3′-Diaminobenzidine (DAB); 3,5-dichloro-2-hydroxybenzenesulfonic acid with 4-aminoantipyrine (DHBS/AAP)), an enzyme (e.g., horseradish peroxidase), and/or a fluorescent species (e.g., a fluorophore).

Reagents may be used in various ways in the article, depending on the desired application. In some embodiments, an article comprises one or more reagents suitable for performing a measurement of a level of hematocrit in blood and/or plasma. For example, the article may comprise: (1) an anti-coagulant, such as ethylenediaminetetraacetic acid; and/or (2) sodium chloride. The reagents may be present within the absorbent layer of the article (e.g., within a sample collection region of an absorbent layer).

In some embodiments, an article comprises one or more reagents suitable for performing a measurement of a level of hemoglobin in blood and/or plasma. For example, the article may comprise: (1) an oxidizing agent, such as ammonium persulfate; (2) a buffer such, as acetate buffer; (3) a reducing agent, such as ascorbic acid; (4) a colorimetric indicator, such as bathophenanthroline, ferrozine, 1,10-phenanthroline, or Drabkin's reagent; (5) a surfactant, such as O,O′-Bis(2-aminopropyl) propylene glycol-block-polyethylene glycol-block-polypropylene glycol (e.g., Jeffamine), poly(diallyldimethylammonium chloride), or polyoxyethylene (23) lauryl ether (e.g., Brij23, Brij 35, C12E23); and/or (6) a cell lysis reagent, such as saponin. When Drabkin's reagent is employed as the colorimetric indicator, the presence of yellow or orange in the article may be indicative of high levels of hemoglobin and/or the presence of red in the sample collection region may be indicative of low levels of hemoglobin. The reagents may be present within the absorbent layer of the article (e.g., within a sample collection region of an absorbent layer). The color of the absorbent layer may be analyzed with a spectrophotometer, a camera (e.g., a camera of a smart-phone), a scanner, or any of a variety of other appropriate colorimetric detectors.

In some embodiments, an article comprises one or more reagents suitable for performing an immunoassay, such as an immunoassay for malaria, HIV, dengue, hCG (e.g., to determine pregnancy), Hepatitis C, C reactive protein (CRP), Vitamin B12, or interferon gamma. For example, the article may comprise: (1) a blocking agent, such as bovine serum album, skim milk powder, and/or casein; (2) a surfactant, such as polyethylene glycol sorbitan monolaurate (e.g., Tween 20); (3) a buffer, such as phosphate buffered saline, a sodium carbonate buffer, and/or a HEPES buffer; (4) a ligand configured to capture a species to be assayed, such as a monoclonal or a polyclonal antibody, a nanobody, and/or an aptamer (which is optionally conjugated to a species that may be easily detected, such as a colored particle (e.g., a colloidal gold nanoparticle), a colorimetric reagent (e.g., 3,3′,5,5′-Tetramethylbenzidine (TMB); potassium iodide (KI); 2,2′-azino-bis(3-ethylbenzothiazoline-6-sulfonic acid (ABTS); or 3,3′-Diaminobenzidine (DAB); 3,5-dichloro-2-hydroxybenzenesulfonic acid with 4-aminoantipyrine (DHBS/AAP)), an enzyme (e.g., horseradish peroxidase), and/or a fluorescent species (e.g., a fluorophore)); and/or (5) a treatment agent and/or a stabilizing agent, such as sucralose, trehalose, and/or albumin. Non-limiting examples of suitable antibodies include anti-pLDH (malaria), anti-p24 (HIV), anti-hCG (pregnancy), anti-CRP (acute phase injury), anti-NS1 (dengue) and anti-human IgG. The reagents may be present within the absorbent layer of the article (e.g., within a sample collection region of an absorbent layer).

In some embodiments, an article comprises one or more reagents suitable for performing an enzymatic assay, such as an enzymatic assay for acetylcholinesterase (e.g., as found on red blood cell membranes) and/or liver enzymes (e.g., alkaline phosphatase, such as found in plasma). For example, the article may comprise: (1) a colorimetric indicator, such as 5,5-dithio-bis-(2-nitrobenzoic acid); (2) an enzymatic substrate, such as acetylthiocholine chloride; and/or (3) a buffer, such as tris buffer. The reagents may be present within the absorbent layer of the article (e.g., within a sample collection region of an absorbent layer).

In some embodiments, an article comprises one or more reagents suitable for performing a blood type analysis. For example, the article may comprise: (1) anti-A sera; (2) anti-B sera; and/or (3) anti-D sera. The reagents may be present within the absorbent layer of the article (e.g., within a sample collection region of an absorbent layer).

In some embodiments, an article comprises one or more reagents suitable for detecting one or more types of cells. For example, the article may comprise: (1) a ligand configured to capture a species to be assayed, such as a monoclonal or a polyclonal antibody, a nanobody, and/or an aptamer (which is optionally conjugated to a species that may be easily detected, such as a colored particle (e.g., a colloidal gold nanoparticle), a colorimetric reagent (e.g., 3,3′,5,5′-Tetramethylbenzidine (TMB); potassium iodide (KI); 2,2′-azino-bis(3-ethylbenzothiazoline-6-sulfonic acid (ABTS); 3,3′-Diaminobenzidine (DAB); 3,5-dichloro-2-hydroxybenzenesulfonic acid with 4-aminoantipyrine (DHBS/AAP)), an enzyme (e.g., horseradish peroxidase), and/or a fluorescent species (e.g., a fluorophore)); and/or (2) a buffer. The reagents may be present within the absorbent layer of the article (e.g., within a sample collection region of an absorbent layer).

An article may comprise one or more reagents suitable for detecting one or more solutes (e.g., one or more solutes in a fluid sample flowing through the article), such as one or more species in a metabolite panel (e.g., glucose, total protein level, alkaline phosphatase, creatinine, low density lipoprotein, high density lipoprotein, triglycerides, and/or blood urea nitrogen), DNA, and/or RNA. For example, the article may comprise: (1) a denaturant configured to act as a stabilizer, such as sodium dodecylsulfate; (2) silk fibroin; (3) RNAse and/or DNAse; and/or (4) an enzyme inhibitor, such as a protease. The reagents may be present within the absorbent layer of the article (e.g., within a sample collection region of an absorbent layer).

The reagents described herein may be present in a variety of suitable locations. In some embodiments, it may be advantageous to include reagents in an absorbent layer. For example, the presence of blocking or stabilizing reagents in the absorbent layer may allow them to retain their interactions with the absorbent layer upon exposure to a fluid (e.g., a blood sample), limiting the ability of cells in the fluid to permanently bind to the absorbent layer and improving recovery. As another example, introducing surfactants or other wetting agents into the absorbent layer may advantageously improve wetting of the sample entering the absorbent layer.

In some embodiments, it may be advantageous to include the reagent in a top layer of the article (e.g., a first filter of the article) so that the reagent rapidly interacts with a fluid (e.g., a blood sample) passed through the article and/or interacts with a fluid before it passes through one or more layers present in the article. For example, it may be advantageous to include pH modifiers, salts, and/or anti-coagulants in the top layer.

Reagents may be present throughout an entire layer (e.g., an entire absorbent layer, an entire filter). Alternatively, a reagent may be introduced only into a portion of a layer (e.g., a sample collection region of an absorbent layer). In some embodiments, all sample collection regions of an absorbent layer include the same reagent. However, in some embodiments, different reagents, or different combinations and/or concentrations of reagents, may be included in different sample collection regions. According to certain embodiments, a configuration where different sample collection regions comprise different reagents may be useful for sample screening.

Within a layer, reagents may be stored in a variety of suitable ways. Non-limiting examples of ways that reagents may be stored in the article include being adsorbed onto a material present in the article (e.g., fibers in an absorbent layer, a material forming a filter), absorbed into a material present in the article (e.g., fibers in an absorbent layer, a material forming a filter), and/or located in a gel present in the article (e.g., in a sample collection region, in a filter, on a filter). In some embodiments, the reagents may be deposited onto one or more fibers in the article (e.g., one or more fibers in an absorbent layer, one or more fibers in a filter). The reagents may be stored in the article as solids. The solids may be present in a matrix, such as a matrix comprising a protein (e.g., BSA) and/or a sugar (e.g., sucralose, trehalose). In some embodiments, one or more reagents stored in an article (e.g., as solids) may be reconstituted and/or dissolved in a fluid (e.g., a blood sample) and/or a portion of a fluid flowing therethrough. For example, a fluid (e.g., a blood sample) and/or a portion of a fluid may flow through a portion of an article comprising one or more reagents, and at least a portion of the one or more reagents may dissolve in the fluid and/or the portion of the fluid as it flows therethrough.

Reagents may be introduced into the articles described herein in a variety of manners. Additionally, reagents may be introduced prior to article assembly or after article assembly. In the former case, reagents may be introduced prior to or after the formation of any barriers or other boundaries (e.g., cuts, perforations, holes) therein. In some embodiments, one or more reagents are introduced into an article by dissolving the reagent(s) in a fluid to produce a reagent solution, exposing the portion(s) of the article to which the reagent(s) are to be introduced to the reagent solution, and subsequently drying the reagent solution so that the reagents are retained in a layer of an article. The resultant dry layer may be subsequently assembled into the article. Drying may be accomplished at room temperature and/or at an elevated temperature (e.g., in a drying oven, at a temperature of 50° C.-65° C.). The drying may occur for periods of time on the order of minutes and/or hours.

In some embodiments, the above-described process is performed sequentially to deposit reagents from two or more solutions that comprise different reagents and/or different combinations of reagents. It is also possible for the above-described process to be performed sequentially to deposit increased amounts of reagents from a single solution.

In some embodiments, the amount of a reagent solution added to a layer and/or the location at which the reagent solution is added may be selected to control the distribution and/or amount of the deposited reagents in the layer. As one example, in some embodiments, a layer is exposed to a low amount of a reagent solution so that a low amount of reagents is deposited in the layer. For instance, a layer may be exposed to a limited amount of a reagent solutions comprising denaturants because the presence of a high concentration of denaturants in a layer may undesirably inhibit plasma permeation through the layer. As another example, in some embodiments, a layer is exposed to a reagent solution at a particular location. For instance, an absorbent layer may be exposed to a reagent solution at a location distal to a filter reception region in order to limit the amount of reagents deposited in the filter reception region and/or proximate thereto. Reagents present in such locations may disadvantageously flow into the filter(s) disposed thereon upon exposure to a fluid flowing through the article (e.g., a blood sample).

Layers containing one or more reagents may comprise the reagent(s) at a variety of appropriate reagent concentrations. For example, in some embodiments, a dry concentration of a reagent in the absorbent layer is greater than or equal to 0.1 mg/cm², greater than or equal to 0.2 mg/cm², greater than or equal to 0.5 mg/cm², greater than or equal to 1 mg/cm², greater than or equal to 2 mg/cm², greater than or equal to 5 mg/cm², or greater. In some embodiments, a dry concentration of a reagent in the absorbent layer is less than or equal to 20 mg/cm², less than or equal to 10 mg/cm², less than or equal to 8 mg/cm², less than or equal to 5 mg/cm², less than or equal to 2 mg/cm², or less. Combinations of these ranges are also possible (e.g., greater than or equal to 0.1 mg/cm² and less than or equal to 20 mg/cm², greater than or equal to 0.1 mg/cm² and less than or equal to 10 mg/cm², greater than or equal to 0.5 mg/cm² and less than or equal to 5 mg/cm²). Any suitable reagent concentration may be used, although appropriate concentration ranges may depend on the type of reagent.

In some embodiments, an article may comprise one or more features designed to aid identification of the article and/or one or more samples contained therein. For instance, the article may comprise a QR code, which may be linked to an online database including one or more types of information, such as information about a patient from which samples on contained on the article have originate and/or information about a hospital and/or clinic used by the patient (and/or at which the article was used to obtain the samples). In some embodiments, a QR code may be used to improve tracking of the article.

The articles described herein may have one or more features of the articles described in the U.S. Provisional Application entitled “Fluidic Articles Involving Signal Generation at Converging Liquid Fronts”, filed on Jun. 22, 2018, incorporated herein by reference in its entirety. The articles described herein may have one or more features of the fluidic articles described in International Patent Publication No. WO 2017/123668, filed on Jul. 20, 2017, and entitled “Separation of Cells Based on Size and Affinity Using Paper Microfluidic Article”, incorporated herein by reference in its entirety.

The following examples are intended to illustrate certain embodiments of the present invention, but do not exemplify the full scope of the invention.

### Example 1

This example compares a variety of exemplary articles comprising circular filter reception regions connected via channels to sample collection regions having a variety of shapes, according to certain embodiments.

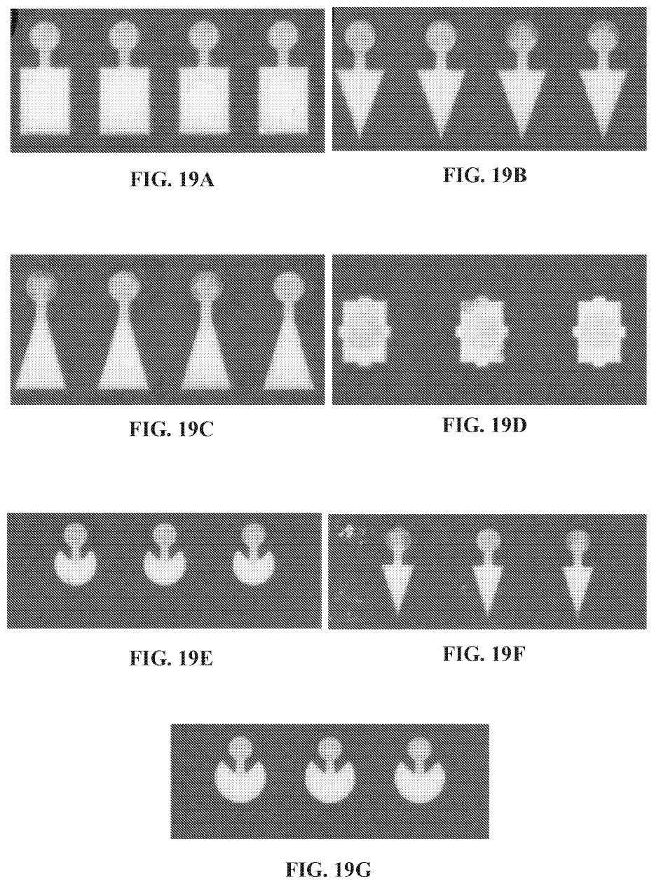

FIGS. 19A-19G provide images of the exemplary articles. Article A comprised a sample collection region having a rectangular form (FIG. 19A). Article B comprised a sample collection region having a triangular form connected to the terminus of the channel at the base of the triangle (FIG. 19B). Article C comprised a sample collection region having a triangular form connected to the terminus of the channel at a vertex of the triangle (FIG. 19C). Article D comprised a sample collection region having a stacked configuration, such that the sample was collected directly below the filters (FIG. 19D). Article E comprised a sample collection region having a form of a circular sector (FIG. 19E). Article F comprised a sample collection region having a triangular form smaller than the triangular form of the sample collection regions in Articles B and C and connected to the terminus of the channel at the base of the triangle (FIG. 19F). Finally, Article G comprised a sample collection region having a form of a circular sector having a larger radius than the first circular sector (FIG. 19G). Of these examples, Article E and Article G had the property that the sample collection region comprised a boundary having a section with a relatively uniform distance from the channel terminus, as described in greater detail above.

In this Example, the volume of blood required to fill Articles A-G was compared. As presented in Table 1, the blood volume required to fill the articles varied with the geometry of the sample collection region. In general, the articles comprising a sample collection region with a triangular form connected to a terminus of a channel at a base of the triangle (Articles B and F) and the article comprising the sample collection region with the form of a circular sector (Article E) required the smallest blood volumes for a given hematocrit level.

TABLE 1
This table presents the estimated volume of whole blood required to fill a sample collection
region of an article, as a function of the hematocrit level (Hct) of the blood. The
hematocrit level of the blood was incremented in 5% increments. Values in bold indicate
that less than 110 microliters of blood was required to fill the device.

| | Blood Volume (microliters) | | | | | | |
|---|---|---|---|---|---|---|---|
| Article | Hct 60% | Hct 55% | Hct 50% | Hct 45% | Hct 40% | Hct 35% | Hct 30% |
| Article A | 279 | 248 | 224 | 203 | 186 | 172 | 160 |
| Article B | 157 | 139 | 125 | 114 | 105 | 97 | 89.6 |
| Article C | 187 | 167 | 150 | 136 | 125 | 115 | 107 |
| Article D | 166 | 148 | 133 | 121 | 111 | 102 | 94.8 |
| Article E | 158 | 140 | 126 | 115 | 105 | 97 | 90.3 |
| Article F | 136 | 121 | 109 | 99 | 90.9 | 84 | 77.9 |
| Article G | 205 | 182 | 164 | 149 | 137 | 126 | 117 |

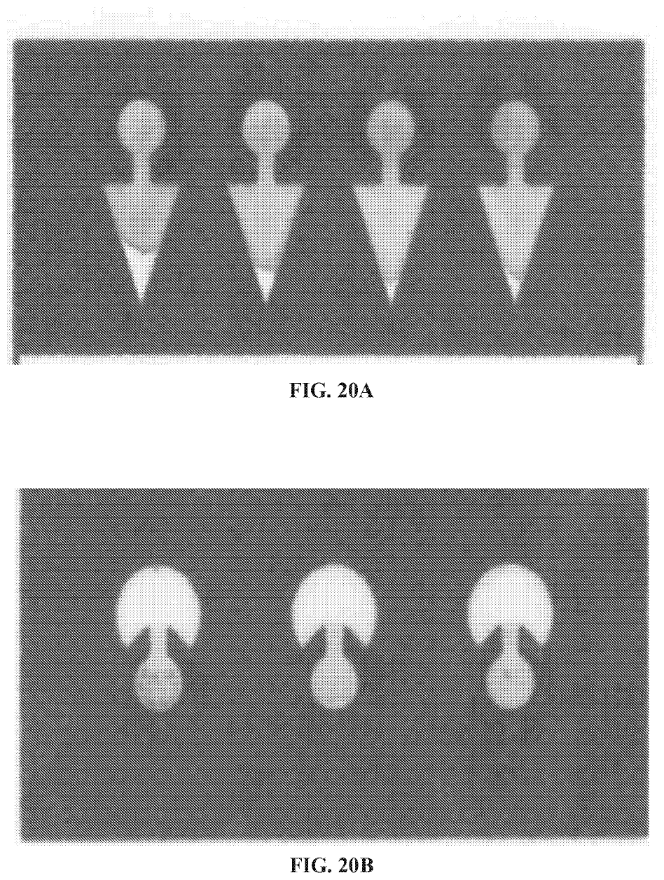

However, when blood with a high hematocrit level was provided to the articles, extensive hemolysis was observed on Article B, while very little hemolysis was observed on Article E. This is apparent from visual inspection of the articles, photographs of which are presented in FIG. 20A (Article B) and FIG. 20B (Article E). As shown, Article B experienced a discoloration (a red discoloration that appears gray in the figure) in the triangular sample collection region, while Article E remained relatively white. This indicates a high degree of hemolysis in Article B, which resulted the collection of discoloring cellular material in the sample collection region along with the plasma.

As this example illustrates, the sample collection region of Article E, which comprised a boundary having a section with a relatively uniform distance from the channel terminus, resulted in the collection of a relatively pure plasma using a relatively low blood volume, demonstrating that the boundary having a section with a relatively uniform distance from the channel terminus may help to control fluid transport through the filter, reducing hemolysis.

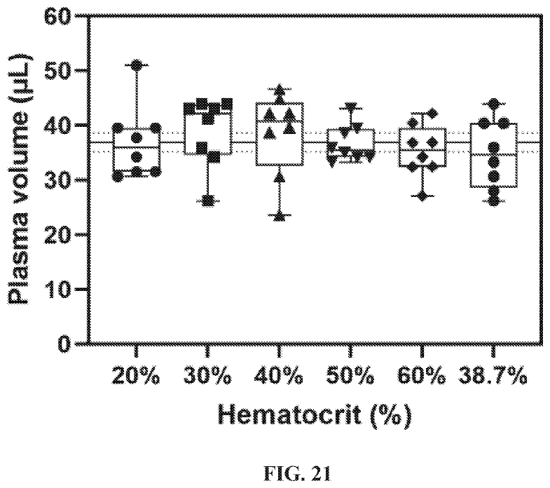

Another unexpected advantage was noted for the design of Article E. As presented in FIG. 21, when a 100 microliter sample was provided to the article, a high plasma yield was extracted regardless of the hematocrit level of the input plasma. Using an ANOVA test, it was determined that no statistically significant difference in the average plasma volume extracted (37±2 microliters) existed at the different hematocrit levels. Without wishing to be bound by theory, this high extraction volume may result from the boundary having a section with a relatively uniform distance from the channel terminus, which allows for plasma to be distributed evenly across the sample collection region.

These examples demonstrate that exemplary articles of the type described herein can separate blood cells from plasma. Furthermore, these examples demonstrate that articles comprising a sample collection region having a boundary having a section with a relatively uniform distance from the channel terminus may perform particularly well, in some embodiments, compared to articles comprising sample collection regions without this property.

### Example 2

This Example demonstrates that the use of reagents in an absorbent layer can improve recovery of low density lipoprotein cholesterol (LDL-C) from plasma introduced thereto.

Reagent-treated absorbent layers were fabricated by exposing sample collection regions within the absorbent layers to 40 microliters of solutions comprising a reagent or a combination of reagents, thereby fully wetting the sample collection regions with the solutions. The reagents and reagent combinations are listed in Table 1. Reagents present in an amount of 5 weight/volume % (w/v %) had a dry reagent density of 1.96 mg/cm² in the sample collection region and reagents present in an amount of 2.5 (w/v %) had a dry reagent density of 0.98 mg/cm² in the sample collection region. A control absorbent layer was left untreated. Then, each reagent-treated absorbent layer and the untreated absorbent layer were affixed to frames and dried under ambient, room-temperature conditions for 90 minutes. The dried layers were then assembled into an exemplary article.

The efficiency of LDL-C recovery from the final, dried reagent-treated and untreated absorbent layers was then determined. Samples of whole blood were separated by centrifugation, and then the resulting purified plasma was applied to the exemplary sample collection regions and the articles were allowed to dry. Finally, plasma was extracted from the dried and plasma-loaded sample collection regions. Additional liquid plasma was used as a liquid control for analytic purposes. LDL-C recovered from the extracted and liquid control plasma was assayed in order to determine its concentration, and the resulting concentration and standard error of the mean LDL-C concentration (SEM) were used to determine a theoretical recovery percentage and coefficient of variation (CV). In general, high recovery and low CV correspond to improved measurement accuracy when extrapolating the LDL-C level in whole blood.

Table 2 presents the results for each of the reagents tried, as well as the LDL-C level from a liquid control. As shown, the blocking agents BSA and skim milk powder improved recovery of the LDL-C in comparison to the untreated absorbent layer, whereas the sugars did not. BSA, in particular, was associated with improved LDL-C recovery. All reagents produced relatively low CVs, with the exception of 2.5% trehalose+5% BSA and 5% skim milk, which produced relatively higher CVs.

TABLE 2
Treatment of collection layer for enhanced recovery of LDL-C.

| | [LDL-C] | SEM | | |
|---|---|---|---|---|
| Sample | (mg/dL) | (mg/dL) | Recovery | CV |
| untreated absorbent | 57.0 | 1.4 | 45% | 2.4% |
| layer | | | | |
| 5 w/v % BSA | 100.9 | 3.0 | 80% | 3.0% |
| 5 w/v % skim milk | 81.0 | 4.4 | 64% | 5.4% |
| 2.5 w/v % trehalose | 55.2 | 1.1 | 44% | 2.0% |
| 2.5 w/v % sucrose | 53.6 | 1.3 | 43% | 2.4% |
| 2.5 w/v % trehalose + | 70.8 | 4.0 | 56% | 5.7% |
| 5 w/v % BSA | | | | |
| 2.5 w/v % sucrose + 5 | 76.4 | 2.1 | 61% | 2.8% |
| w/v % skim milk | | | | |
| liquid control | 126.1 | 2.0 | 100% | 1.6% |

Additional experiments were performed to determine whether the presence of any of the reagents listed in Table 2 in a separation device/absorbent layer would affect the ability to determine the LDL-C concentration in a tested sample. Samples for analysis were prepared by assaying each of the reagents and reagent combinations listed in Table 2 for apparent signal from LDL-C. The signal that would ordinarily correspond to the LDL-C concentration was measured before and after the addition of the reagent or reagent combination to the other components of the assay. The changes in signal are shown in Table 3, which reports each change in signal in terms of an apparent change in LDL-C concentration, and the changes were observed to be negligible for each reagent.

TABLE 3
Interference of reagents for the quantification of LDL-C.

| | Change in [LDL-C] |
|---|---|
| Sample | (mg/dL) |
| untreated absorbent layer | N/A |
| 5 w/v % BSA | 0.4 |
| 5 w/v % skim milk | 1.1 |
| 2.5 w/v % trehalose | −1.0 |
| 2.5 w/v % sucrose | −0.4 |
| 2.5 w/v % trehalose + 5 w/v % BSA | 0.5 |
| 2.5 w/v % sucrose + 5 w/v % skim milk | 0.4 |
| liquid control | N/A |

These results demonstrate that blocking agents may improve recovery of plasma analytes, and demonstrates that the inclusion of reagents does not interfere with quantification of the analytes.

### Example 3

This example describes filtering of plasma from a blood sample using non-limiting articles comprising varying numbers of sample collection regions (1, 2, 3, or 4). Each article was designed to have a total volume equaling approximately 40 percent of the volume of the total volume of the blood sample to be applied to the article (150 microliters), with the volume being split between the number of sample collection regions used in the article.

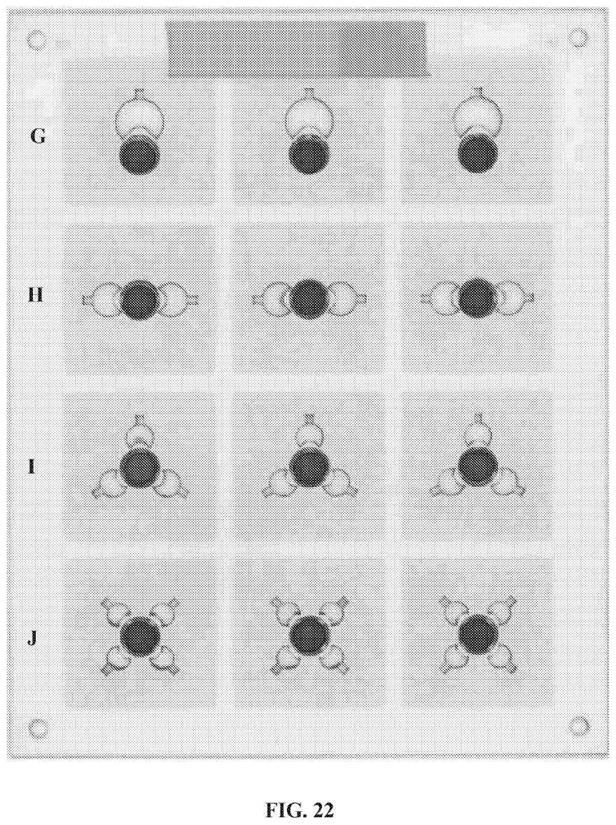

FIG. 22 presents a photograph of the non-limiting articles used to separate 150 microliter blood volumes. The articles used to separate 150 microliter blood volumes included Articles G (comprising one sample collection region), Articles H (comprising two sample collection regions), Articles I (comprising three sample collection regions), and Articles J (comprising 4 sample collection regions). A 150 microliter blood sample was added to a filter of each article (dark regions), which passed the filtered sample into the sample collection region(s) (white regions). Very little visual discoloration of the plasma collection regions was observed, indicating a high degree of purity in the plasma collected. After collection, the articles were dried, and dry mass of the plasma in each zone was measured.

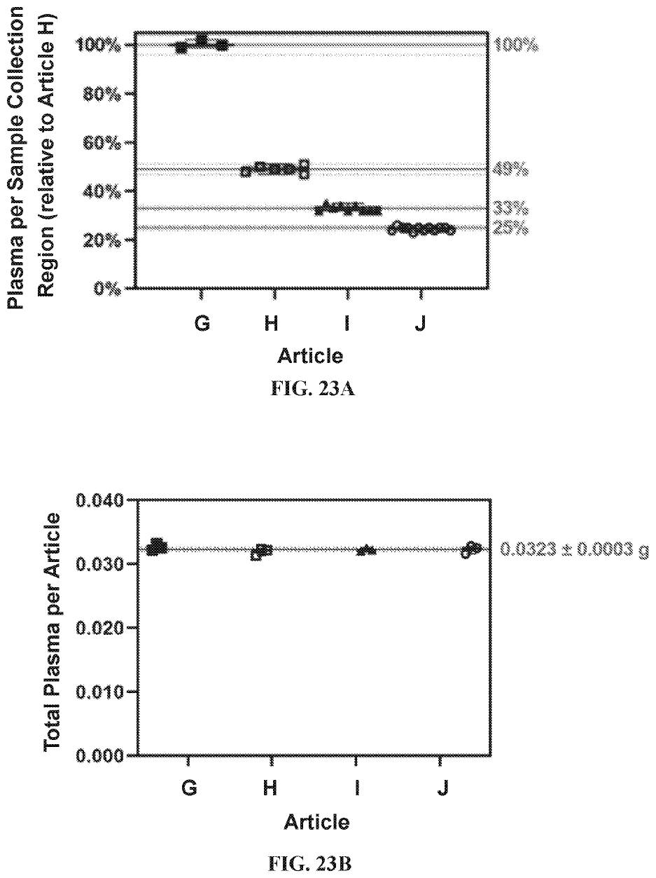

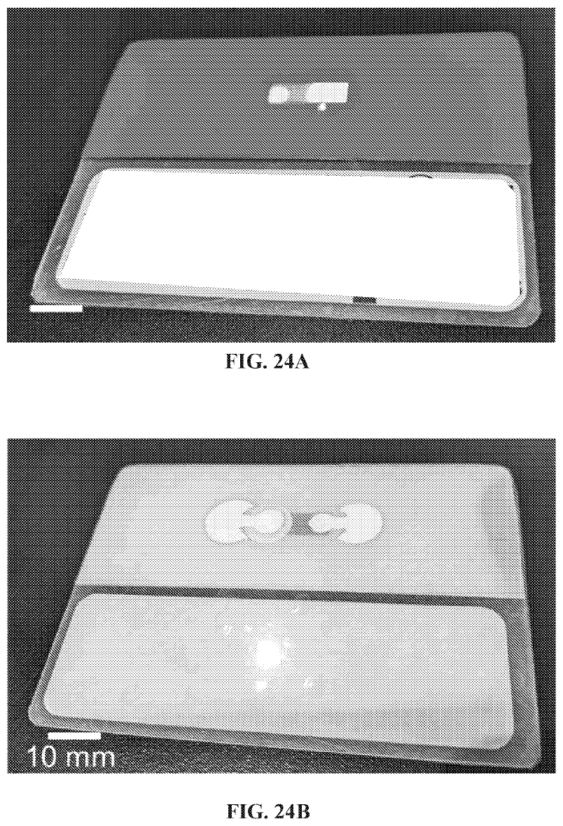

FIG. 23A presents the dry plasma mass stored within each sample collection region of Articles G-H versus the total number of sample collection regions in the article. Since 3 replicates of each article were used, there were 3 measurements for Articles G, 6 for Articles H, 9 for Articles I, and 12 for Articles J, corresponding to the total number of sample collection regions that could be measured. The Y axis represents the average plasma mass per sample collection region, normalized by the average plasma mass of the sample collection regions of Articles G. The reciprocal relationship between number of sample collection regions and average plasma mass per sample collection region indicates demonstrates that the filtered plasma is divided evenly between the sample collection regions. This is further evidenced by FIG. 24, which totals the average mass of recovered plasma per article.

This example demonstrates that quantitative recovery of plasma can be performed using articles comprising multiple sample collection regions, and demonstrates that the total mass recovery of filtered plasma does not depend on the number of sample collection regions used.

### Example 4

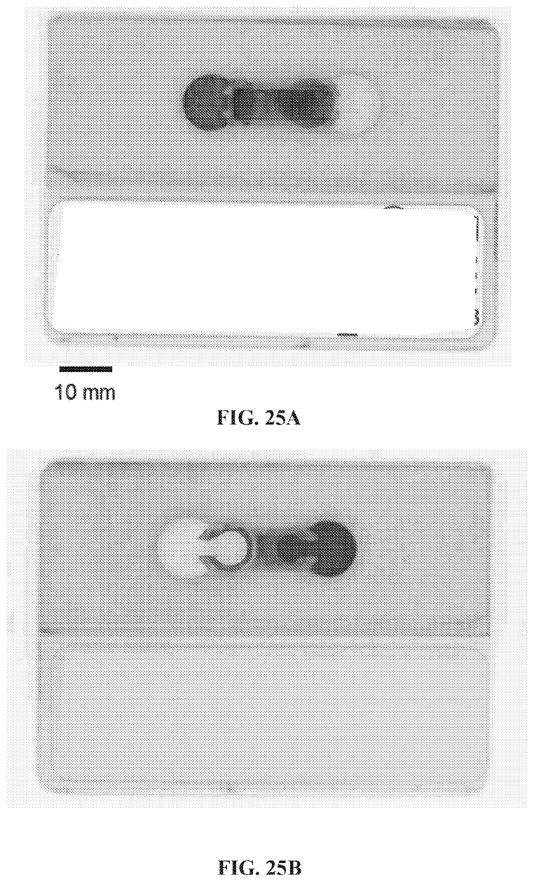

This example describes simultaneous recovery of plasma and whole blood from a whole blood sample using an article comprising a fluid distribution layer, according to some embodiments. The article had the structure described in FIG. 12A above. FIG. 24A presents a photograph of the front of the article prior to use, while FIG. 24B presents a photograph of the back of the article prior to use, wherein the sample collection region (left and the whole blood collection region (right) can clearly be seen. Next, a 140 microliter blood sample was added to the inlet at the front of the article. FIG. 25A presents a front-view of the resulting article, and FIG. 25B presents a back-view of the resulting article. As shown, the non-limiting ultimately included a filled whole blood collection region and a filled (plasma) sample collection region, demonstrating that such an article could successfully separate whole blood and filtered plasma into separate zones for subsequent analysis.

### Example 5

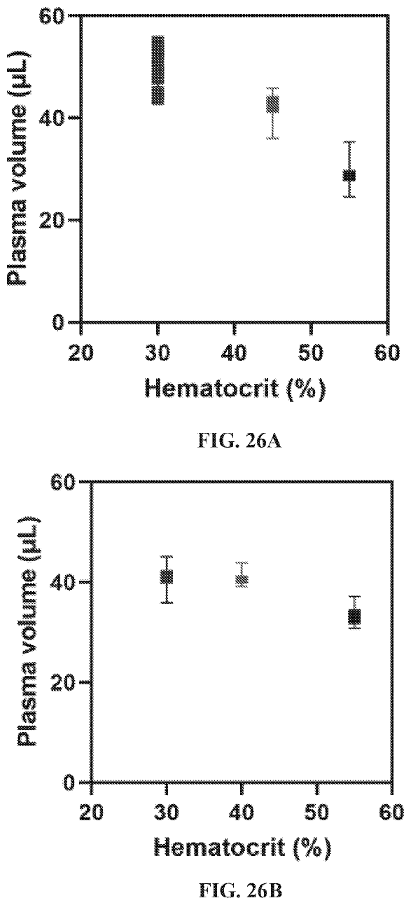

This example describes recovery of plasm using non-limiting articles comprising sample collection regions with and without a second, absorption layer comprising an overflow region disposed beneath the sample collection regions. The articles were constructed as shown in FIG. 14 (with or without the overflow region). In this example, testing was performed on blood samples with varying hematocrit levels ranging between 30% and 60%. FIG. 26A presents the plasma volume collected in the sample collection region of the article without the overflow region as a function of hematocrit level. FIG. 26B presents the plasma volume collected in the sample collection region of the article with the overflow region as a function of hematocrit level.

As shown in FIG. 26A, the plasma volume stored in the recovery region depended significantly on the hematocrit percentage of the blood itself, with low-hematocrit blood filling the sample collection region more fully and more inconsistently. In contrast, when the overflow region was added, the plasma volume reached an effective maximum value of 40 microliters, and the collected volume included significantly lower sample variance. This example demonstrates that overflow regions can be used to improve control of the plasma volume collected by the sample collection region of an article, which may be advantageous for performing accurate assays.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, “wt %” is an abbreviation of weight percentage. As used herein, “at %” is an abbreviation of atomic percentage.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An article configured to separate blood cells from plasma, comprising:
a first filter configured to retain blood cells;
a second filter configured to retain blood cells, wherein the second filter is disposed beneath the first filter, and wherein the first and second filters are positioned such that a sample comprising separated blood cells can be recovered therefrom; and
an absorbent layer comprising a porous, absorbent material, wherein:
the absorbent layer is disposed beneath the second filter,
the absorbent layer comprises a sample collection region fluidically connected with and laterally spaced from the second filter, and
the sample collection region is configured to receive plasma from which blood cells have been separated from the second filter.

2. An article configured to separate blood cells from plasma, comprising:
a filter configured to retain blood cells; and
an absorbent layer comprising a porous, absorbent material, wherein:
the absorbent layer is disposed beneath the filter,
the absorbent layer comprises a sample collection region laterally spaced from the filter,
the absorbent layer comprises a channel fluidically connecting the filter to the sample collection region,
the sample collection region is configured to receive plasma from which blood cells have been separated, and
the sample collection region is laterally bounded in a plane of the channel by a boundary and a terminus of the channel,
the boundary comprises a section having a distance from the terminus of the channel, and
a standard deviation of a distance from the terminus of the channel to the section is less than or equal to 30% of an average distance from a terminus of the channel to the section, and wherein the section makes up greater than or equal to 15% of the boundary.

3-4. (canceled)

5. An article as in claim 1, wherein the first filter is configured to separate white blood cells and/or leukocytes from plasma.

6. An article as in claim 1, wherein the second filter is configured to separate red blood cells and/or platelets from plasma.

7. An article as in claim 1, wherein at least a portion of the absorbent layer has an absorbency of greater than or equal to 14 microliters/cm² and less than or equal to 120 microliters/cm².

8. An article as in claim 1, wherein the first filter is porous and has a mode pore size that is greater than or equal to 1 micron and less than or equal to 30 microns.

9. An article as in claim 1, wherein the second filter is porous and has a mode pore size that is greater than or equal to 0.1 micron and less than or equal to 5 microns.

10. An article as in claim 1, wherein the second filter is porous and greater than or equal to 20% of the pores of the second filter have a pore size of less than or equal to 20 microns.

11. (canceled)

12. An article as in claim 1, wherein the absorbent layer is configured to transport fluid via capillary action.

13. (canceled)

14. An article as in claim 1, wherein the sample collection region has the shape of a sector of a circle.

15. An article as in claim 1, wherein the absorbent layer comprises a channel fluidically connecting the second filter to the sample collection region, wherein the sample collection region is laterally bounded in a plane of the channel by a boundary and a terminus of the channel, and wherein at least a section of the boundary is a gap in the absorbent layer.

16. An article as in claim 1, wherein the absorbent layer comprises a channel fluidically connecting the second filter to the sample collection region, wherein the sample collection region is laterally bounded in a plane of the channel by a boundary and a terminus of the channel, and wherein at least a section of the boundary is an external boundary of the absorbent layer.

17-18. (canceled)

19. An article as in claim 1, wherein the absorbent layer comprises a channel fluidically connecting the second filter to the sample collection region, wherein the sample collection region is laterally bounded in a plane of the channel by a boundary and a terminus of the channel, and wherein at least a section of the boundary of the sample collection region is perforated.

20. An article as in claim 1, wherein the sample collection region is configured to be removed from the article using tweezers.

21. An article as in claim 1, wherein the first filter comprises a polyester.

22. An article as in claim 1, wherein the second filter comprises an asymmetric polysulfone.

23. An article as in claim 1, wherein the first filter and/or the second filter is removable from the article.

24. An article as in claim 1, wherein the first filter separates blood cells based on size exclusion and electrostatic interactions.

25-26. (canceled)

27. An article as in claim 1, wherein the article comprises a reagent or a combination of reagents, and wherein the reagent or combination of reagents comprises a blocking reagent, a stabilizing reagent, a surfactant, a denaturant, a salt, an anti-coagulant, and/or a pH modifier.

28-30. (canceled)

31. A method, comprising:
passing a blood sample comprising blood cells and plasma to an absorbent layer through a first filter and a second filter to separate at least a portion of the blood cells from the plasma; and
transporting the plasma laterally within the absorbent layer to a sample collection region that is laterally spaced from the first and second filters.

32-103. (canceled)
